(19) United States
(12) Patent Application Publication
(10) Pub. No.: US 2010/0022646 A1
(43) Pub. Date: Jan. 28, 2010

(54) METHOD OF NEUTRALISING ORGANOBORONATES WITH ACIDS

(76) Inventors: Vincent Patric Russel, Slough (GB); Suresh Babubhai Chahwala, London (GB); Shouming Wang, London (GB)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/399,289

(22) Filed: Mar. 6, 2009

Related U.S. Application Data
(63) Continuation of application No. 11/292,419, filed on Nov. 30, 2005, now abandoned.
(60) Provisional application No. 60/632,782, filed on Dec. 2, 2004.

(30) Foreign Application Priority Data
Nov. 30, 2004 (GB) 0426264.8

(51) Int. Cl. A61K 31/20

(52) U.S. Cl. 514/560

(57) ABSTRACT

The use of specified compounds for the manufacture of a medicament for therapeutically neutralising an organoboronate drug. The specified compounds are typically hydroxy fatty acids or hydroperoxy fatty acids, for example 9(S)-HODE, 8(S)-HETRE or 8(S)-HEPE, or their salts or prodrugs. The organoboronate drug may be TRI 50c or a salt or prodrug thereof. Also disclosed are intravenous formulations containing the specified compounds.

### CROSS-REFERENCE

This application claims the benefit of Great Britain Patent Application No. 0426264.8, filed on Nov. 30, 2004, and U.S. Provisional Application No. 60/632,782, filed Dec. 2, 2004, which applications are incorporated herein by reference in their entirety.

### BACKGROUND

The present disclosure relates to inhibitors of biologically active organoboronates and more particularly of organoboronate medicaments and enzyme inhibitors; the enzymes are more particularly serine proteases, e.g. serine protease anticoagulants. The disclosure additionally includes compounds having a pharmacophore described herein; it further includes a class of fatty acids and fatty acid derivatives. The disclosure also relates to the use of members of the aforesaid products, to their formulation, and to other subject matter.

### Boronic Acid Compounds

It has been known for some years that boronic acid compounds and their derivatives, e.g. esters, have biological activities, notably as inhibitors or substrates of proteases. For example, Koehler et al. Biochemistry 10:2477, 1971 report that 2-phenylethane boronic acid inhibits the serine protease chymotrypsin at millimolar levels. The inhibition of chymotrypsin and subtilisin by arylboronic acids (phenylboronic acid, m-nitro-phenylboronic acid, m-aminophenylboronic acid, m-bromophenylboronic acid) is reported by Phillip et al, Proc. Nat. Acad. Sci. USA 68:478-480, 1971. A study of the inhibition of subtilisin Carlsberg by a variety of boronic acids, especially phenyl boronic acids substituted by Cl, Br, CH₃, H₂N, MeO and others, is described by Seufer-Wasserthal et al, Biorg. Med. Chem. 2(1):35-48, 1994.

In describing inhibitors or substrates of proteases, P1, P2, P3, etc. designate substrate or inhibitor residues which are amino-terminal to the scissile peptide bond, and S1, S2, S3, etc., designate the corresponding subsites of the cognate protease in accordance with: Schechter, I. and Berger, A. On the Size of the Active Site in Proteases, Biochem.Biophys.Res.Comm., 27:157-162, 1967. In thrombin, the S1 binding site or “specificity pocket” is a well defined groove in the enzyme, whilst the S2 and S3 binding subsites (also respectively called the proximal and distal hydrophobic pockets) are hydrophobic and interact strongly with, respectively, Pro and (R)-Phe, amongst others.

Pharmaceutical research into serine protease inhibitors has moved from the simple arylboronic acids to boropeptides, i.e. peptides containing a boronic acid analogue of an α-amino carboxylic acid. The boronic acid may be derivatised, often to form an ester. Shenvi (EP-A-145441 and U.S. Pat. No. 4,499,082) disclosed that peptides containing an α-aminoboronic acid with a neutral side chain were effective inhibitors of elastase and has been followed by numerous patent publications relating to boropeptide inhibitors of serine proteases. Specific, tight binding boronic acid inhibitors have been reported for elastase (Kᵢ, 0.25 nM), chymotrypsin (Kᵢ, 0.25 nM), cathepsin G (Kᵢ, 21 nM), α-lytic protease (Kᵢ, 0.25 nM), dipeptidyl aminopeptidase type IV (Kᵢ, 16 pM) and more recently thrombin (Ac-D-Phe-Pro-boroArg-OH (DuP 714 initial Kᵢ 1.2 nM).

Claeson et al (U.S. Pat. No. 5,574,014 and others) and Kakkar et al (WO 92/07869 and family members including U.S. Pat. No. 5,648,338) disclose thrombin inhibitors having a neutral C-terminal side chain, for example an alkyl or alkoxyalkyl side chain.

Modifications of the compounds described by Kakkar et al are included in WO 96/25427, directed to peptidyl serine protease inhibitors in which the P2-P1 natural peptide linkage is replaced by another linkage. As examples of non-natural peptide linkages may be mentioned —CO₂—, —CH₂O—, —NHCO—, —CHYCH₂—, —CH═CH—, —CO(CH₂)ₚCO— where p is 1, 2 or 3, —COCHY—, —CO₂—CH₂NH—, —CHY—NX—, —N(X)CH₂—N(X)CO—, —CH═C(CN)CO—, —CH(OH)—NH—, —CH(CN)—NH—, —CH(OH)—CH₂— or —NH—CHOH—, where X is H or an amino protecting group and Y is H or halogen, especially F. Particular non-natural peptide linkages are —CO₂— or —CH₂O—.

Metternich (EP 471651 and U.S. Pat. No. 5,288,707) discloses variants of Phe-Pro-BoroArg boropeptides in which the P3 Phe is replaced by an unnatural hydrophobic amino acid such as trimethylsilylalanine, p-tert.butyl-diphenyl-silyloxymethyl-phenylalanine or p-hydroxymethylphenylalanine and the P1 side chain may be neutral (alkoxyalkyl, alkylthioalkyl or trimethylsilylalkyl).

The replacement of the P2 Pro residue of borotripeptide thrombin inhibitors by an N-substituted glycine is described in Fevig J M et al Bioorg. Med. Chem. 8: 301-306 and Rupin A et al Thromb. Haemost. 78(4):1221-1227, 1997. See also U.S. Pat. No. 5,585,360 (de Nanteuil et al).

Amparo (WO 96/20698 and family members including U.S. Pat. No. 5,698,538) discloses peptidomimetics of the structure Aryl-linker-Boro(Aa), where Boro(Aa) may be an aminoboronate residue with a non-basic side chain, for example BoroMpg. The linker is of the formula —(CH₂)ₘCONR— (where m is 0 to 8 and R is H or certain organic groups) or analogues thereof in which the peptide linkage —CONR— is replaced by —CSNR—, —SO₂NR—, —CO₂—, —C(S)O— or —SO₂O—. Aryl is phenyl, naphthyl or biphenyl substituted by one, two or three moieties selected from a specified group. Most typically these compounds are of the structure Aryl-(CH₂)ₙ—CONH—CHR²—BY¹Y², where R² is for example a neutral side chain as described above and n is 0 or 1.

Non-peptide boronates have been proposed as inhibitors of proteolytic enzymes in detergent compositions. WO 92/19707 and WO 95/12655 report that arylboronates can be used as inhibitors of proteolytic enzymes in detergent compositions. WO 92/19707 discloses compounds substituted meta to the boronate group by a hydrogen bonding group, especially acetamido (—NHCOCH₃), sulfonamido (—NHSO₂CH₃) and alkylamino. WO 95/12655 teaches that ortho-substituted compounds are superior.

Boronate enzyme inhibitors have wide application, from detergents to bacterial sporulation inhibitors to pharmaceuticals. In the pharmaceutical field, there is patent literature describing boronate inhibitors of serine proteases, for example thrombin, factor Xa, kallikrein, elastase, plasmin as well as other serine proteases like prolyl endopeptidase and Ig AI Protease. Thrombin is the last protease in the coagulation pathway and acts to hydrolyse four small peptides from each molecule of fibrinogen, thus deprotecting its polymerisation sites. Once formed, the linear fibrin polymers may be cross-linked by factor XIIIa, which is itself activated by thrombin. In addition, thrombin is a potent activator of platelets, upon which it acts at specific receptors. Thrombin also potentiates its own production by the activation of factors V and VIII.

Other aminoboronate or peptidoboronate inhibitors or substrates of serine proteases are described in:
U.S. Pat. No. 4,935,493
EP 341661
WO 94/25049
WO 95/09859
WO 96/12499
WO 96/20689
Lee S-L et al, Biochemistry 36:13180-13186, 1997
Dominguez C et al, Bioorg. Med. Chem. Lett. 7:79-84, 1997
EP 471651
WO 94/20526
WO 95/20603
WO 97/05161
U.S. Pat. No. 4,450,105
U.S. Pat. No. 5,106,948
U.S. Pat. No. 5,169,841.

Peptide boronic acid inhibitors of hepatic C virus protease are described in WO 01/02424. Matteson D S Chem. Rev. 89: 1535-1551, 1989 reviews the use of α-halo boronic esters as intermediates for the synthesis of inter alia amino boronic acids and their derivatives. Matteson describes the use of pinacol boronic esters in non-chiral synthesis and the use of pinanediol boronic esters for chiral control, including in the synthesis of amino and amido boronate esters.

Boronic acid and ester compounds have displayed promise as inhibitors of the proteasome, a multicatalytic protease responsible for the majority of intracellular protein turnover. Ciechanover, Cell, 79:13-21, 1994, teaches that the proteasome is the proteolytic component of the ubiquitin-proteasome pathway, in which proteins are targeted for degradation by conjugation to multiple molecules of ubiquitin. Ciechanover also teaches that the ubiquitin-proteasome pathway plays a key role in a variety of important physiological processes.

Adams et al, U.S. Pat. No. 5,780,454 (1998), U.S. Pat. No. 6,066,730 (2000), U.S. Pat. No. 6,083,903 (2000) and equivalent WO 96/13266, and U.S. Pat. No. 6,297,217 (2001) describe peptide boronic ester and acid compounds useful as proteasome inhibitors. These documents also describe the use of boronic ester and acid compounds to reduce the rate of muscle protein degradation, to reduce the activity of NF-κB in a cell, to reduce the rate of degradation of p53 protein in a cell, to inhibit cyclin degradation in a cell, to inhibit the growth of a cancer cell, to inhibit antigen presentation in a cell, to inhibit NF-κB dependent cell adhesion, and to inhibit HIV replication. Brand et al, WO 98/35691, teaches that proteasome inhibitors, including boronic acid compounds, are useful for treating infarcts such as occur during stroke or myocardial infarction. Elliott et al, WO 99/15183, teaches that proteasome inhibitors are useful for treating inflammatory and autoimmune diseases.

A proteasome inhibitor disclosed in the Adams et al patents is bortezomib (Velcade®), the compound N-(2-pyrazine)-carbonyl-phenylalanine-leucine-boronic acid.

WO 02/059131 discloses boronic acid products which are certain boropeptides and/or boropeptidomimetics in which the boronic acid group has been derivatised with a sugar. The disclosed sugar derivatives, which have hydrophobic amino acid side chains, are of the formula

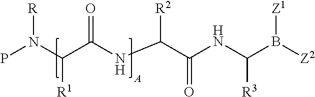

wherein:
P is hydrogen or an amino-group protecting moiety;
R is hydrogen or alkyl;
A is 0, 1 or 2;
R¹, R² and R³ are independently hydrogen, alkyl, cycloalkyl, aryl or —CH₂—R⁵;
R⁵, in each instance, is one of aryl, aralkyl, alkaryl, cycloalkyl, heterocyclyl, heteroaryl, or —W—R⁶, where W is a chalcogen and R⁶ is alkyl;
where the ring portion of any of said aryl, aralkyl, alkaryl, cycloalkyl, heterocyclyl, or heteroaryl in R¹, R², R³ or R⁵ can be optionally substituted; and
Z¹ and Z² together form a moiety derived from a sugar, wherein the atom attached to boron in each case is an oxygen atom.

Some of the disclosed compounds are sugar derivatives of bortezomib (see above), e.g. its mannitol ester.

### Thrombosis

Hemostasis is the normal physiological condition of blood in which its components exist in dynamic equilibrium. When the equilibrium is disturbed, for instance following injury to a blood vessel, certain biochemical pathways are triggered leading, in this example, to arrest of bleeding via clot formation (coagulation). Coagulation is a dynamic and complex process in which proteolytic enzymes such as thrombin play a key role. Blood coagulation may occur through either of two cascades of zymogen activations, the extrinsic and intrinsic pathways of the coagulation cascade. Factor VIIa in the extrinsic pathway, and Factor IXa in the intrinsic pathway are important determinants of the activation of factor X to factor Xa, which itself catalyzes the activation of prothrombin to thrombin, whilst thrombin in turn catalyses the polymerization of fibrinogen monomers to fibrin polymer. The last protease in each pathway is therefore thrombin, which acts to hydrolyze four small peptides (two FpA and two FpB) from each molecule of fibrinogen, thus deprotecting its polymerization sites. Once formed, the linear fibrin polymers may be cross-linked by factor XIIIa, which is itself activated by thrombin. In addition, thrombin is a potent activator of platelets, upon which it acts at specific receptors. Thrombin activation of platelets leads to aggregation of the cells and secretion of additional factors that further accelerate the creation of a hemostatic plug. Thrombin also potentiates its own production by the activation of factors V and VIII (see Hemker and Beguin in: Jolles, et. al., “Biology and Pathology of Platelet Vessel Wall Interactions,” pp. 219-26 (1986), Crawford and Scrutton in: Bloom and Thomas, “Haemostasis and Thrombosis,” pp. 47-77, (1987), Bevers, et. al., Eur. J. Biochem. 122:429-36, 1982, Mann, Trends Biochem. Sci. 12:229-33, 1987).

Proteases are enzymes which cleave proteins at specific peptide bonds. Cuypers et al., J. Biol. Chem. 257:7086, 1982, and the references cited therein, classify proteases on a mechanistic basis into five classes: serine, cysteinyl or thiol, acid or aspartyl, threonine and metalloproteases. Members of each class catalyse the hydrolysis of peptide bonds by a similar mechanism, have similar active site amino acid residues and are susceptible to class-specific inhibitors. For example, all serine proteases that have been characterised have an active site serine residue.

The coagulation proteases thrombin, factor Xa, factor VIIa, and factor IXa are serine proteases having trypsin-like specificity for the cleavage of sequence-specific Arg-Xxx peptide bonds. As with other serine proteases, the cleavage event begins with an attack of the active site serine on the scissile bond of the substrate, resulting in the formation of a tetrahedral intermediate. This is followed by collapse of the tetrahedral intermediate to form an acyl enzyme and release of the amino terminus of the cleaved sequence. Hydrolysis of the acyl enzyme then releases the carboxy terminus.

A thrombus can be considered as an abnormal product of a normal mechanism and can be defined as a mass or deposit formed from blood constituents on a surface of the cardiovascular system, for example of the heart or a blood vessel. Thrombosis can be regarded as the pathological condition wherein improper activity of the hemostatic mechanism results in intravascular thrombus formation.

The management of thrombosis commonly involves the use of antiplatelet drugs (inhibitors of platelet aggregation) to control future thrombogenesis and thrombolytic agents to lyse the newly formed clot, either or both such agents being used in conjunction or combination with anticoagulants. Anticoagulants are used also preventatively (prophylactically) in the treatment of patients thought susceptible to thrombosis.

### Neutral P1 Residue Boropeptide Thrombin Inhibitors

Claeson et al (U.S. Pat. No. 5,574,014 and others) and Kakkar et al (WO 92/07869 and family members including U.S. Pat. No. 5,648,338) disclose lipophilic thrombin inhibitors having a neutral (uncharged) C-terminal (P1) side chain, for example an alkoxyalkyl side chain.

The Claeson et al and Kakkar et al patent families disclose boronate esters containing the amino acid sequence D-Phe-Pro-BoroMpg [(R)-Phe-Pro-BoroMpg], which are highly specific inhibitors of thrombin. Of these compounds may be mentioned in particular Cbz-(R)-Phe-Pro-BoroMpg-OPinacol (also known as TRI 50b). The corresponding free boronic acid is known as TRI 50c. For further information relating to TRI 50b and related compounds, the reader is referred to the following documents:
Elgendy S et al., in The Design of Synthetic Inhibitors of Thrombin, Claeson G et al Eds, Advances in Experimental Medicine, 340:173-178, 1993.
Claeson G et al, Biochem J. 290:309-312, 1993
Tapparelli C et al, J Biol Chem, 268:4734-4741, 1993
Claeson G, in The Design of Synthetic Inhibitors of Thrombin, Claeson G et al Eds, Advances in Experimental Medicine, 340:83-91, 1993
Phillip et al, in The Design of Synthetic Inhibitors of Thrombin, Claeson G et al Eds, Advances in Experimental Medicine, 340:67-77, 1993
Tapparelli C et al, Trends Pharmacol. Sci. 14:366-376, 1993
Claeson G, Blood Coagulation and Fibrinolysis 5:411-436, 1994
Elgendy et al, Tetrahedron 50:3803-3812, 1994
Deadman J et al, J. Enzyme Inhibition 9:29-41, 1995
Deadman J et al, J. Medicinal Chemistry 38:1511-1522, 1995.

The tripeptide sequence of TRI 50b has three chiral centres. The Phe residue is considered to be of (R)-configuration and the Pro residue of natural (S)-configuration, at least in compounds with commercially useful inhibitor activity; the Mpg residue is believed to be of (R)-configuration in isomers with commercially useful inhibitor activity. TRI 50b acts as a prodrug for corresponding free acid TRI 50c, which is the active principle. The active, or most active, TRI 50c stereoisomer is considered to be of R,S,R configuration and may be represented as:

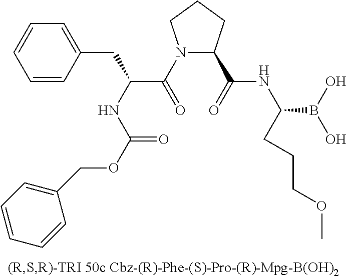

WO 2004/022072, and also U.S. Ser. No. 10/659,178 and EP-A-1396270, disclose pharmaceutically acceptable base addition salts of boronic acids which have a neutral aminoboronic acid residue capable of binding to the thrombin S1 subsite linked through a peptide linkage to a hydrophobic moiety capable of binding to the thrombin S2 and S3 subsites. In a first embodiment, there is disclosed a pharmaceutically acceptable base addition salt of a boronic acid of, for example, formula (A):

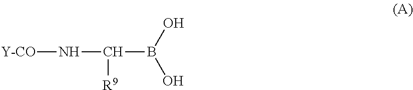

wherein

Y comprises a hydrophobic moiety which, together with the aminoboronic acid residue —NHCH(R⁹)—B(OH)₂, has affinity for the substrate binding site of thrombin; and

R⁹ is a straight chain alkyl group interrupted by one or more ether linkages (e.g. 1 or 2) and in which the total number of oxygen and carbon atoms is 3, 4, 5 or 6 (e.g. 5) or R⁹ is —(CH₂)ₘ—W where m is 2, 3, 4 or 5 (e.g. 4) and W is —OH or halogen (F, Cl, Br or I). R⁹ is an alkoxyalkyl group in one subset of compounds, e.g. alkoxyalkyl containing 4 carbon atoms. Salts of TRI 50c are exemplary.

WO 2004/022071, and also U.S. Ser. No. 10/659,179 and EP-A-1396269, disclose salts of a pharmaceutically acceptable multivalent (at least divalent) metal and an organoboronic acid drug. Such salts are described as having an improved level of stability which cannot be explained or predicted on the basis of known chemistry, and as being indicated to have unexpectedly high and consistent oral bioavailability not susceptible of explanation on the basis of known mechanisms. The oral formulations of such salts are therefore also disclosed.

One particular class of salts comprises those wherein the organoboronic acid comprises a boropeptide or boropeptidomimetic. Such drugs which may beneficially be prepared as salts include without limitation those of the formula X-(aa)ₙ-B(OH)₂, where X is H or an amino-protecting group, n is 2, 3 or 4, (especially 2 or 3) and each aa is independently a hydrophobic amino acid, whether natural or unnatural. In one class of multivalent metal salts, the organoboronic acid is of formula (A) above. Salts of TRI 50c are exemplary.

WO 2004/022070, and also U.S. Ser. No. 10/658,971 and EP-A-1400245, disclose and claim inter alia parenteral pharmaceutical formulations that include a pharmaceutically acceptable base addition salt of a boronic acid of, for example, formula (A) above. Such salts are described as having an improved level of stability which cannot be explained or predicted on the basis of known chemistry. Salts of TRI 50c are exemplary.

### Non-Peptide Boronates

Non-peptide boronates have been proposed as inhibitors of proteolytic enzymes in detergent compositions. WO 92/19707 and WO 95/12655 report that arylboronates can be used as inhibitors of proteolytic enzymes in detergent compositions. WO 92/19707 discloses compounds substituted meta to the boronate group by a hydrogen bonding group, especially acetamido (—NHCOCH₃), sulfonamido (—NHSO₂CH₃) and alkylamino. WO 95/12655 teaches that ortho-substituted compounds are superior.

Potential Bleeding Resulting from Use of Anticoagulants

When an anticoagulant is used as a therapeutic agent, there can be a requirement for rapid neutralisation should there be unexpected bleeding. The problem of unwanted bleeding is well recognised in the area of anticoagulant therapy; thus protamine is used to reverse the anticoagulant effect of heparin and vitamin K to reverse the effect of warfarin.

### BRIEF SUMMARY OF THE DISCLOSURE

The present invention relates amongst other things to products useful for neutralising (reducing or destroying) the activity of biologically active organoboronates. Unless the context otherwise requires, the term “boronate” as used herein includes reference to boronic acids as well as derivatised forms thereof, e.g. salts as well as esters and other prodrugs. In one embodiment, the present disclosure provides a product that can neutralise the activity of a boropeptidyl serine protease inhibitor and thus terminate or reduce its therapeutic effect if required. The term “neutralise” as used herein includes both reduction of activity and destruction of activity. In the pharmaceutical industry, compounds having such activities are known as “antidotes”, “reversal agents” and “neutralisers”, and doubtless by other names also.

Included in the disclosure is the use, for the manufacture of a medicament for therapeutically neutralising (e.g. reducing in activity) an organoboronate drug, of a compound selected from organic acids comprising an aliphatic moiety substituted by a nucleophilic group and salts and prodrugs of such acids. The acid may contain up to 30 carbon atoms, for example; in one class of organic acids, there are at least 6 carbon atoms, e.g. at least 12 carbon atoms. Aliphatic acids are particularly to be mentioned. Said acid is not a boron acid, e.g. a boronic or borinic acid.

The nucleophilic group may be a hydroxy, hydroperoxy or amino group.

The aliphatic moiety may contain a fragment —C*(H)A- where C* is a chiral centre of (S)-configuration and A is a nucleophilic group. Also included are compounds containing a fragment —C(H)A- at the α-position to a carbon-carbon double bond, for example compounds containing a fragment —C(H)A-CH═C(R^{a})(H) where R^{a} comprises, e.g. is, an aliphatic group. This aliphatic group may be hydrocarbyl or 1, 2, 3, 4 or more hydrogens of the hydrocarbyl group may be replaced by a substituent such as a hydroxy or another nucleophilic group, for example. The aliphatic group may be saturated or it may contain one or more carbon-carbon multiple bonds, particularly carbon-carbon double bonds. The aliphatic group may be entirely open-chain, e.g. a linear or branched group, as in the case of an alkyl group, an alkoxyalkyl group or an alkenyl group containing 1, 2, 3, 4 or more double bonds; in one class of compounds such groups form the entirety of R^{a}. R^{a} may contain for example from 2 to 15 in-chain and/or in-ring atoms (usually these are all aliphatic; often they are all carbon), e.g. 5 to 10 in-chain and/or in-ring atoms and often 7, 8, 9 or 10 in-chain and/or in-ring atoms, as in the case of an alkenyl group containing 1, 2 or 3 double bonds and 7, 8 or 9 carbon atoms. R^{a} may be a linear hydrocarbyl group optionally substituted by one or more nucleophilic groups. Please see below under Formula (III) under the heading “DETAILED DESCRIPTION OF SEVERAL EXAMPLES” for further examples of R^{a} groups.

The organoboronate neutralised by the above-described compound may be a protease inhibitor; it may be a serine protease inhibitor, e.g. a coagulation serine protease inhibitor, for example TRI 50c, or a proteasome inhibitor, for example bortezomib. The disclosure is not limited as to whether the organoboronate is administered in the form of its active principle or in the form of a derivative, for example a salt, sugar adduct or prodrug thereof: all such possibilities are included.

The disclosed compounds, therefore, are administered as pharmaceutical formulations, e.g. oral or parenteral. As particular parenteral formulations may be mentioned intravenous formulations. Included in the disclosure are intravenous formulations comprising a compound containing a pharmacophore (or structural fragment) of Formula (III), or a salt or prodrug thereof:

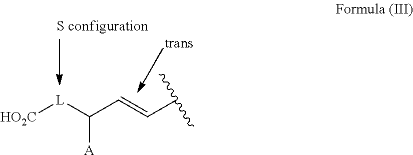

wherein:

A is a nucleophilic group; and

L is a linker containing from 5 to 10 in-chain atoms, e.g. 7 or 8.

A is hydroxy in a particular class of compounds. As other possible A groups may be mentioned hydroperoxy and amino.

The in-chain atoms of L may by way of example be C, O, N or S, e.g. C or O. Included is a class of formulations in which all the in-chain atoms of L are carbon. The in-chain bonds of L are typically double or single bonds; included is a class of compounds in which all the in-chain bonds are single bonds or are all single except one double bond.

The remainder of the molecule to which the fragment of Formula (III) is bonded may be an aliphatic group and is a linear aliphatic group in one class of compounds. Included in the disclosure is a class of compounds in which the remainder of the molecule is an R^{a} moiety as described above.

Exemplary are 18C and 20C fatty acids containing the above pharmacophore. The fatty acids may be substituted by at least one further nucleophilic functional group, for example.

The compounds may be in the form of prodrugs or salts thereof. The prodrugs may be esters. References herein to acids should be construed to include salt, protected and prodrug forms unless the context requires otherwise. Salt forms may in principle comprise any pharmaceutically acceptable salt, and may in particular include the sodium salt.

The present disclosure also includes subject matter of the following paragraphs:

1. A method for therapeutically neutralising (i.e. reducing or substantially destroying the activity of) an organoboronate drug in a subject, comprising administering to the subject a therapeutically useful amount of a compound selected from organic acids other than boron acids comprising an aliphatic moiety substituted by a nucleophilic group, and salts and prodrugs of such acids.

2. The method of paragraph 1 wherein the acid contains up to 30 carbon atoms.

3. The method of paragraph 1 or paragraph 2 wherein the acid contains at least 12 carbon atoms.

4. The method of paragraph 3 wherein the acid contains 12, 13, 14, 15, 16, 17, 18, 19 or 20 carbon atoms.

5. The method of any preceding paragraph wherein the nucleophilic group is a hydroxy, hydroperoxy or amino group.

6. The method of any preceding paragraph wherein the acid contains a plurality of the nucleophilic groups.

7. The method of any preceding paragraph wherein the aliphatic moiety contains a fragment —C*(H)A- where C* is a chiral centre of (S)-configuration and A is a said nucleophilic group.

8. The method of paragraph 7 wherein the acid contains a fragment —C(H)A- at the α-position to a carbon-carbon double bond.

9. The method of paragraph 8 wherein fragment —C(H)A- and the carbon-carbon double bond together form a fragment —C(H)A-CH═C(R^{a})(H) where R^{a} is an aliphatic moiety optionally substituted by one or more substituents selected from A groups, halogen, alkyl, alkoxy, haloalkyl and carboxy.

10. The method of paragraph 9 wherein the double bond shown in the fragment is of trans (E) configuration and is optionally conjugated to a cis (Z) double bond within moiety R^{a}.

11. The method of any preceding paragraph wherein the acid comprises an alkane or an alkene containing 1, 2 or 3 double bonds and substituted by nucleophilic and acid moieties.

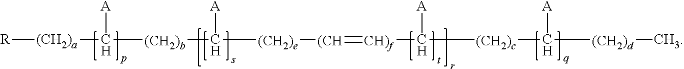

12. The method of paragraph 1 wherein the acid is of the formula (I):

where:

R is —COOH, —P(O)(OH)₂, —HPOOH, or —SO₃H;

A is a nucleophilic group;

the sum of a and b is an integer from 4 to 9 inclusive and a or b may be 0;

the sum of c and d is an integer from 1 to 6 inclusive and c or d may be 0

e and f are independently 0 or 1;

r is an integer from 1 to 6; and

p, q, s and t are independently 0 or 1, the value of each instance of s and t being independent of the value of each other instance (if any) and there being at least one —C(H)A- moiety; and at least one —CH₂— group within the compound may be replaced by an ether linkage —O— or an amine linkage —N—.

13. The method of paragraph 12 in which the acid comprises at least one —CH═CH— moiety, i.e. in which at least one instance of f is 1.

14. The method of paragraph 13 in which the —CH═CH— moiety is directly bonded to a —C(H)A- moiety.

15. The method of paragraph 14 in which the —CH═CH— moiety is in trans (E) configuration.

16. The method of paragraph 12 in which the acid comprises a fragment —C(H)A-CH═CH— in which A is a hydroxy group

17. The method of paragraph 16 in which the double bond is of trans configuration.

18. The method of paragraphs 16 or 17 in which the hydroxy group is separated from the acid group (R in formula (I)) by 7 or 8 atoms.

19. The method of paragraph 12 in which a+p+b is at least 5 and the moiety R—(CH₂)a-[C(H)A]ₚ—(CH₂)_{b}— is directly bonded to a moiety C(H)A-CH═CH—.

20. The method of paragraph 19 in which a+p+b is from 5 to 10.

21. The method of paragraph 19 or paragraph 20 in which the double bond may be in trans (E) configuration.

22. The method of any of paragraphs 19 to 21 in which the double bond is conjugated to a double bond in cis (Z) configuration.

23. The method of any one of paragraphs 19 to 23 in which the cis double bond is not further conjugated to a further double bond.

24. The method of any of paragraphs 19 to 23 in which moiety R—(CH₂)a-[C(H)A]ₚ—(CH₂)_{b}— includes an ether linkage —O— or an amine linkage —N— in place of a —CH₂— group.

25. The method of any of paragraphs 12 to 24 in which s and t are 0 in at least one fragment —[C(H)A]ₛ-(CH₂)ₑ—(CH═CH)_{f}—[C(H)A]ₜ—.

26. The method of paragraph 25 in which p+q is 1.

27. The method of paragraphs 12 to 26 in which f is 1 and e either 0 or 1 in at least one fragment —[C(H)A]ₛ-(CH₂)ₑ—(CH═CH)_{f}-[C(H)A]ₜ—.

28. The method of paragraphs 12 to 27 in which r is 2, 3 or 5.

29. The method of any of paragraphs 12 to 28 in which the acid of Formula (I) has from 6 to 24 carbon atoms in total, at least one of the carbon atoms optionally being replaced by an oxygen or nitrogen atom.

30. The method of paragraph 29 in which the acid has from 14 to 20 carbon atoms

31. The method of any of paragraphs 12 to 30 in which the acid contains 1 or 2 nucleophilic groups A.

32. The method of paragraphs 12 to 31 in which the acid has an R group which is —COOH or a salt or an ester thereof.

33. The method of paragraphs 12 to 32 in which the or each A is —OH, —OOH or —NH₂ the identity of any one A group being independent from that of any other A groups.

34. The method of paragraph 33 in which the or each A is —OH or —OOH.

35. The method of paragraph 34 in which the or each A is —OH.

36. The method of any of paragraphs 12 to 35 in which any one or more hydrogen atoms of the acid (I) is replaced by a halogen.

37. The method of paragraph 1 in which the acid is of the Formula (II):

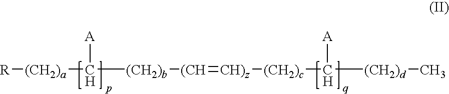

where:

R is —COOH, —P(O)(OH)₂, —HPOOH, or —SO₃H;

A is a nucleophilic group;

the sum of a and b is an integer from 4 to 9 inclusive and a or b may be 0;

the sum of c and d is an integer from 1 to 6 inclusive and c or d may be 0

z is 1 or 2; and

p and q are independently 0 or 1 and p+q is at least 1.

38. The method of paragraph 37 in which p is 1, b is 0 and z is 1 or 2.

39. The method of paragraph 37 or paragraph 38 in which the acid has a C═C moiety directly bonded to —[C(H)A]-.

40. The method of paragraph 39 in which the double bond is of trans configuration and any double bond to which it is conjugated is of cis configuration.

41. The method of paragraph 1 in which the acid comprises a pharmacophore (or structural fragment) of Formula (III):

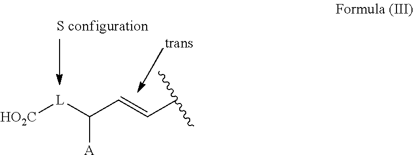

wherein:

A is a nucleophilic group; and

L is a linker containing from 5, 6, 7, 8, 9 or 10 in-chain atoms.

42. The method of paragraph 41 in which L contains 7 or 8 in-chain atoms.

43. The method of paragraph 41 or 42 in which A is hydroxy.

44. The method of paragraph 41 or 42 in which A is hydroperoxy or amino.

45. The method of paragraphs 41 to 44 in which the in-chain atoms of L are C, O, N or S.

46. The method of paragraph 45 in which the in-chain atoms are C or O.

47. The method of paragraph 45 in which the in-chain atoms are C.

48. The method of paragraph 45 in which the in-chain bonds are double or single bonds.

49. The method of paragraph 45 in which all the in-chain bonds are single bonds.

50. The method of paragraph 45 in which the in-chain bonds comprise at least one carbon-carbon double bond.

51. The method of any one of paragraphs 41-50 wherein the linker L is unsubstituted.

52. The method of any one of paragraphs 41-50 wherein the linker L is substituted.

53. The method of any one of paragraphs 41-50 or 52 in which each in-chain atom has at least one substituent.

54. The method of any one of paragraphs 41-50 or 52 in which L contains fewer substituents than in-chain atoms.

55. The method of any one of paragraphs 41-50 or 52 in which no in-chain atom contains more than one substituent.

56. The method of any of paragraphs 41 to 55 in which in which L has 1, 2 or 3 substituents.

57. The method of paragraph 56 in which L has 1 substituent.

58. The method of any of paragraphs 41-50 or 52-57, wherein the substituents are selected from A groups, halogen (e.g. F or Cl), 1C to 4C alkyl, 1C to 4C alkoxy, 1C to 4C haloalkyl and carboxy.

59. The method of paragraph 58 wherein the substituents on L are selected from halogen or an A group.

60. The method of paragraph 58 wherein at least one substituent is hydroxy.

61. The method of paragraph 1 in which the acid comprises a pharmacophore of Formula (IV):

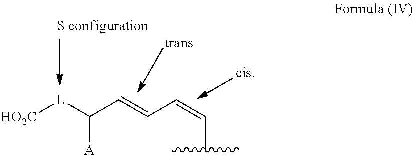

62. The method of paragraph 1 in which the acid comprises a pharmacophore of formula (V) where the cis (Z) double bond is not conjugated to a second trans double bond:

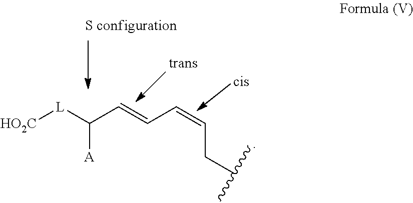

63. The method of any of paragraphs 41 to 62 wherein the acid is of the formula (XV):

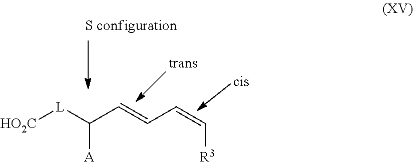

where R³ is a linear aliphatic moiety.

64. The method of paragraph 1 in which the acid comprises a pharmacophore of formula (VI):

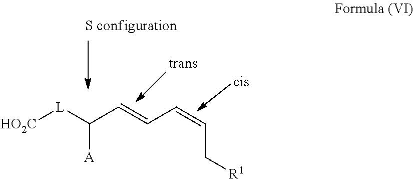

where R¹ is H or 1C-10C alkyl or 2C-10C alkenyl, optionally substituted by one or more substituents, e.g. 1, 2, 3 or 4 substituents selected from A groups, halogen (e.g. F or Cl), 1C to 4C alkyl, 1C to 4C alkoxy, 1C to 4C haloalkyl and carboxy.
65. The method of paragraph 64 wherein the one or more optional substituents on R¹ are selected from halogen and A groups.

66. The method of paragraph 63 in which R¹ is unsubstituted.

67. The method of any of paragraphs 63 to 65 in which R¹ is substituted by at least one substituent.

68. The method of paragraph 67 in which R¹ contains fewer substituents than in-chain atoms.

69. The method of paragraph 67 or paragraph 68 in which no in-chain atom contains more than one substituent.

70. The method of any of paragraphs 67 to 69 in which R¹ has 1, 2, 3 or 4 substituents.

71. The method of any of paragraphs 63 to 70 in which R¹ is alkyl and has 3, 4, 5, 6, 7 or 8 carbon atoms.

72. The method of paragraphs 63 to 70 in which R¹ is alkenyl and has one or more double bonds.

73. The method of paragraph 72 wherein R¹ has a plurality of double bonds, at least some of the double bonds being conjugated.

74. The method of paragraph 72 wherein R¹ has a plurality of double bonds, the double bonds being unconjugated.

75. The method of paragraph 73 wherein two double bonds are conjugated.

76. The method of paragraph 1 in which the acid comprises a pharmacophore of formula (VII):

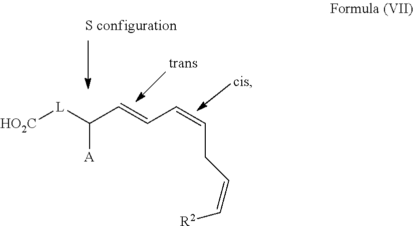

where R² is a residue of an R¹ group corresponding to the fragment R²—CH═CH—, e.g. is H or 1C to 8C alkyl or 2C to 8C alkenyl, for example 4C, 5C or 6C alkyl or alkenyl.

77. The method of paragraph 1 in which the acid is 9(S)-hydroxy-10E,12Z-octadecadienoic acid.

78. The method of paragraph 1 in which the acid is 8(S)-hydroxy-9E, 11Z, 14Z-eicosatrienoic acid.

79. The method of paragraph 1 in which the acid is 8(S)-hydroxy-5Z, 9E, 11Z, 14Z, 17Z-eicosapentaenoic acid.

80. The method of any one of paragraphs 1 to 79 where the acid is in the form of a salt or an ester.

81. The method of any preceding paragraph wherein the boronate group of the organoboronate drug is bonded to an aliphatic carbon atom.

82. The method of any preceding paragraph wherein the boronate group of the organoboronate drug is bonded to an sp³ carbon atom.

83. The method of any preceding paragraph wherein the organoboronate drug is a peptide boronate.

84. The method of paragraph 83 wherein the peptide boronate has a C-terminal residue which is of an α-aminoboronic acid having an alkyl or alkoxyalkyl side chain.

85. The method of paragraph 84 wherein the C-terminal residue is of Boro-3-methoxypropylglycine.

86. The method of paragraph 85 wherein the drug is Cbz-(R)-Phe-(S)-Pro-(R)-Mpg-B(OH)₂, whether administered as the free acid, a salt or a prodrug.

87. An intravenous formulation comprising a compound as defined in any one of paragraphs 41 to 80.

88. An intravenous formulation of paragraph 87 which is for use in neutralising an aminoboronate drug.

89. An intravenous formulation of paragraph 87 or 88 which is for use in treating bleeding resulting from administration of an aminoboronate inhibitor of a serine protease.

90. An intravenous formulation of paragraph 89 wherein the aminoboronate inhibitor has a neutral P1 side chain.

91. An intravenous formulation of paragraph 90 which is for use in treating bleeding resulting from administration of an aminoboronate of formula (IX):

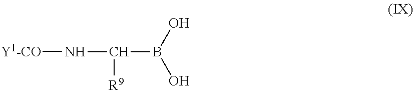

or a salt or prodrug thereof, wherein peptide linkage(s) in the aminoboronate of formula (X) are substituted or unsubstituted and:

Y¹ comprises a hydrophobic moiety which, together with the aminoboronic acid residue —NHCH(R⁹)—B(OH)₂, has affinity for the substrate binding site of thrombin; and

R⁹ is a straight chain alkyl group interrupted by one or more ether linkages and in which the total number of oxygen and carbon atoms is 3, 4, 5 or 6 or R⁹ is —(CH₂)ₘ—W where m is 2, 3, 4 or 5 and W is —OH or halogen.

92. An intravenous formulation of paragraph 91 wherein the aminoboronate is an optionally N-terminally protected tripeptide having the amino acid sequence Phe-Pro-BoroMpg.

93. An intravenous formulation of paragraph 92 wherein the tripeptide sequence has (R,S,R)-configuration.

94. An intravenous formulation of any of paragraphs 87 to 93 which includes a pharmaceutically acceptable diluent, excipient or carrier.

95. An intravenous formulation of any of paragraphs 87 to 94 wherein the acid is selected from 9(S)-hydroxy-10E,12Z-octadecadienoic acid, 8(S)-hydroxy-9E, 11Z, 14Z-eicosatrienoic acid or 8(S)-hydroxy-5Z, 9E, 11Z, 14Z, 17Z-eicosapentaenoic acid, substantially free of other isomers thereof.

96. A method for reversing the activity of a boropeptide drug in a subject, comprising administering to the subject a therapeutically useful amount of a hydroxy fatty acid or hydroperoxy fatty acid or salt or prodrug thereof for the manufacture of a medicament.

97. The method of paragraph 96 wherein the boropeptide drug is a boropeptidyl serine protease inhibitor.

98. The method of paragraph 96 or 97 wherein the boropeptide drug is a thrombin inhibitor which has a neutral P1 side chain.

99. The method of paragraph 96 wherein the acid is an acid as defined in any of paragraphs 41 to 80 and the boropeptidyl serine protease inhibitor is of Formula (X)

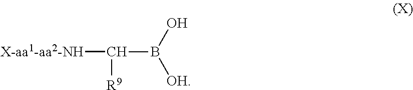

or a salt or prodrug thereof
where:
X is H (to form NH₂) or an amino-protecting group;
aa¹ is Phe, Dpa or a wholly or partially hydrogenated analogue thereof;
aa² is an imino acid having from 4 to 6 ring members; and
R⁹ is a straight chain alkyl group interrupted by one or more ether linkages and in which the total number of oxygen and carbon atoms is 3, 4, 5 or 6 or R⁹ is —(CH₂)ₘ—W where m is 2, 3, 4 or 5 and W is —OH or halogen.

100. The method of paragraphs 96 to 99 wherein the boropeptidyl serine protease inhibitor is an optionally N-terminally protected tripeptide having the amino acid sequence Phe-Pro-BoroMpg.

101. The method of paragraphs 96 to 100 wherein the tripeptide sequence has (R,S,R)-configuration.

102. The method of any of paragraphs 96 to 101 in which the fatty acid is a hydroxylinoleic acid or a hydroperoxylinoleic acid.

103. The method of a compound as defined in any of paragraphs 1 to 80 for the manufacture of a medicament for treating bleeding resulting from the administration of a boropeptide inhibitor of a coagulation serine protease.

104. The method of paragraph 103 wherein the boropeptidyl serine protease inhibitor is an optionally N-terminally protected tripeptide having the amino acid sequence Phe-Pro-BoroMpg.

105. A method for terminating or reducing the activity of a boropeptide drug in a subject, comprising administering to the subject a therapeutically useful amount of a hydroxy fatty acid or hydroperoxy fatty acid or salt or prodrug thereof.

106. A method for terminating or reducing the activity of a boropeptidyl serine protease inhibitor, comprising administering to the subject a therapeutically useful amount of a hydroxy fatty acid or hydroperoxy fatty acid or salt or prodrug thereof.

107. The method of paragraph 105 or paragraph 106 wherein the boropeptidyl serine protease inhibitor comprises the amino acid sequence Phe-Pro-BoroMpg.

108. The method of any of paragraphs 105 to 107 wherein the hydroxy fatty acid or hydroperoxy fatty acid is a hydroxylinoleic acid or perhydroxylinoleic acid.

109. A method of neutralising a boropeptidyl serine protease inhibitor comprising contacting said boropeptidyl serine protease inhibitor with a compound as defined in any of paragraphs 1 to 80.

110. A method of paragraph 109 which is an ex vivo method.

111. A method for at least reducing the activity of an organoboronate drug in a subject, comprising administering to the subject an effective amount of a compound as defined in any of paragraphs 1 to 80.

112. A method for treating bleeding following administration of a boropeptide inhibitor of a coagulation serine protease to an individual, the method comprising administering to the individual a therapeutically effective amount of a compound as described in any of paragraphs 41 to 80.

113. A method according to paragraph 112 wherein the product is administered in an amount such that there is a molar equivalence ratio of approximately 1:1 with boropeptidyl serine protease inhibitor in the individual's plasma.

114. A method of preparing to supply a first pharmaceutical composition for the treatment of thrombosis by prophylaxis or therapy and, if required, a second pharmaceutical composition to inhibit the action of the first composition, comprising:
stocking a pharmaceutical composition comprising a pharmaceutically acceptable active compound which is capable of providing in the plasma a peptide boronic acid of formula (XII); and
stocking a pharmaceutical formulation comprising a compound as defined in any of paragraphs 41 to 80;
formula (XII) being as follows:

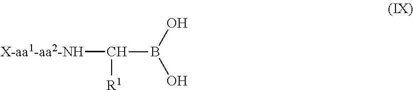

where:
X is H (to form NH₂) or an amino-protecting group;
aa¹ is Phe, Dpa or a wholly or partially hydrogenated analogue thereof;
aa² is an imino acid having from 4 to 6 ring members; and
R¹ is a group of the formula —(CH₂)ₘ—W, where m is 2, 3 or 4 and W is —OH, —OMe, —OEt or halogen (F, Cl, Br or I).

115. A method of providing a medicament pair comprising a first medicament for the treatment of thrombosis by prophylaxis or therapy and a second medicament to inhibit the action of the first medicament in the event of undue bleeding, comprising:
providing a pharmaceutical composition comprising a compound as recited in paragraph 114, and
providing a pharmaceutical composition comprising a compound as defined in any of paragraphs 41 to 80.

116. A method for treating thrombosis by prophylaxis or therapy using a medicament which results in inappropriate bleeding and then inhibiting the action of said medicament, wherein:
a therapeutically effective amount of a pharmaceutical composition comprising a compound as recited in paragraph 92 is administered to a patient in need thereof to treat thrombosis, and, after the inappropriate bleeding,
a therapeutically effective amount of a compound as defined in any of paragraphs 41 to 80 is administered to the patient to inhibit the pharmaceutical composition.

117. A method for inhibiting thrombosis in the treatment of disease by prophylaxis or therapy using a medicament which results in inappropriate bleeding and then inhibiting the action of said medicament, wherein
a therapeutically effective amount of a pharmaceutical composition comprising a compound as recited in paragraph 108 is administered to a patient in need thereof to treat thrombosis, and, after the inappropriate bleeding,
a therapeutically effective amount of a compound as defined in any of paragraphs 41 to 80 is administered to the patient to inhibit the pharmaceutical composition comprising a compound as recited in paragraph 114.

118. A method of providing a medicament pair comprising a first medicament for the treatment of thrombosis by prophylaxis or therapy and a second medicament to inhibit the action of the first medicament in the event of undue bleeding, comprising:
providing a pharmaceutical composition comprising a compound as recited in paragraph 114, and
providing a pharmaceutical composition comprising a compound as defined in any of paragraphs 41 to 80.

119. The use of a compound as defined in any of paragraphs 41 to 80 for the manufacture of a medicament pair comprising a first medicament for treating thrombosis by prophylaxis or therapy and a second medicament for, if required, stopping or reducing the anti-thrombotic treatment, of a compound as recited in paragraph 114 for the manufacture of the first medicament and a compound as defined in any of paragraphs 41 to 80 for the manufacture of the second medicament.

120. A method of preparing for the administration to a patient of a first pharmaceutical composition for the treatment of thrombosis by prophylaxis or therapy and, if required, a second pharmaceutical composition for reacting with the active agent of the first composition to inactivate molecules thereof comprising
supplying a pharmaceutical composition comprising a compound as recited in paragraph 114, and
supplying a pharmaceutical formulation comprising a compound as defined in any of paragraphs 41 to 80.

Further aspects and embodiments of the disclosure are set forth in the following description and claims. Also included as such are all the neutraliser compounds described herein and pharmaceutical formulations containing them.

Throughout the description and claims of this specification, the words “comprise” and “contain” and variations of the words, for example “comprising” and “comprises”, mean “including but not limited to”, and are not intended to (and do not) exclude other moieties, additives, components, integers or steps.

### BRIEF DESCRIPTION OF THE DRAWINGS

FIG. 1 is a graph showing the effect on the anti-coagulant activity of TRI 50c (sodium salt) of various cholesterol linoleic acids;

FIG. 2 is a graph showing the effect on the anti-coagulant activity of TRI 50c (sodium salt) of various cholesterol linoleic acids and of various free linoleic acids; and

FIG. 3 is a graph showing the effect on the anti-coagulant activity of TRI 50c (sodium salt) of particular linoleic acids and derivatives according to the disclosure.

### DETAILED DESCRIPTION OF SEVERAL EXAMPLES

### Glossary

The following terms and abbreviations are used in this specification:

The term “aliphatic” refers to an open-chain or cyclic species not having aromatic properties. Such species may contain a combination of open-chain and cyclic parts. They may be saturated or unsaturated. Often “aliphatic” refers to open-chain species, whether linear or branched, linear being more common. Aliphatic species may be hydrocarbyl aliphatic. Aliphatic species are often substituted or unsubstituted alkyl, alkenyl or alkynyl; in many instances aliphatic is unsubstituted alkyl, alkenyl or alkynyl, e.g. is alkyl. The species may be a compound or part of a compound, as the context requires. Some aliphatic species contain from 1 to 15 in-chain or in-ring atoms, e.g. 1 to 10 such as 1 to 6, for example.

α-Aminoboronic acid or Boro(aa) refers to an amino acid in which the CO₂ group has been replaced by BO₂.

The term “amino-group protecting moiety” refers to any group used to derivatise an amino group, especially an N-terminal amino group of a peptide or amino acid. Such groups include, without limitation, alkyl, acyl, alkoxycarbonyl, aminocarbonyl, and sulfonyl moieties. However, the term “amino-group protecting moiety” is not intended to be limited to those particular protecting groups that are commonly employed in organic synthesis, nor is it intended to be limited to groups that are readily cleavable.

The term “coagulation serine protease” refers to a serine protease involved in the coagulation of blood, for example thrombin, Factor IXa or Factor X.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

The expression “thrombin inhibitor” refers to a product which, within the scope of sound pharmacological judgement, is potentially or actually pharmaceutically useful as an inhibitor of thrombin, and includes reference to substance which comprises a pharmaceutically active species and is described, promoted or authorised as a thrombin inhibitor. Such thrombin inhibitors may be selective, that is they are regarded, within the scope of sound pharmacological judgement, as selective towards thrombin in contrast to other proteases; the term “selective thrombin inhibitor” includes reference to substance which comprises a pharmaceutically active species and is described, promoted or authorised as a selective thrombin inhibitor.

The term “heteroaryl” refers to a ring system which has at least one (e.g. 1, 2 or 3) in-ring heteroatoms and has a conjugated in-ring double bond system. The term “heteroatom” includes oxygen, sulfur and nitrogen, of which sulfur is sometimes less preferred.

“Natural amino acid” means an L-amino acid (or residue thereof) selected from the following group of neutral (hydrophobic or polar), positively charged and negatively charged amino acids:

Hydrophobic Amino Acids
A=Ala=alanine
V=Val=valine
I=Ile=isoleucine
L=Leu=leucine
M=Met=methionine
F=Phe=phenylalanine
P=Pro=proline
W=Trp=tryptophan

Polar (Neutral or Uncharged) Amino Acids
N=Asn=asparagine
C=Cys=cysteine
Q=Gln=glutamine
G=Gly=glycine
S=Ser=serine
T=Thr=threonine
Y=Tyr=tyrosine

Positively Charged (Basic) Amino Acids
R=Arg=arginine
H=H is =histidine
K=Lys=lysine

Negatively Charged Amino Acids
D=Asp=aspartic acid
E=Glu=glutamic acid.

Amino acid=α-amino acid

Acid addition salt=a reaction product made by combining an inorganic acid or an organic acid with a free base of an active principle (e.g. an amino group).

Base addition salt=a reaction product made by combining an inorganic base or an organic base with a free acid (e.g. a carboxylic or boronic acid) of an active principle.

Cbz=benzyloxycarbonyl

Cha=cyclohexylalanine (a hydrophobic unnatural amino acid)

Charged (as applied to drugs or fragments of drug molecules, e.g. amino acid residues)=carrying a charge at physiological pH, as in the case of an amino, amidino or carboxy group

Dcha=dicyclohexylalanine (a hydrophobic unnatural amino acid)

Dpa=diphenylalanine (a hydrophobic unnatural amino acid)

Drug=a pharmaceutically useful substance, whether the active in vivo principle or a prodrug i.v.=intravenous

Mpg=3-methoxypropylglycine (a hydrophobic unnatural amino acid)

Neutral (as applied to drugs or fragments of drug molecules, e.g. amino acid residues)=uncharged=not carrying a charge at physiological pH

Pinac=Pinacol=2,3-dimethyl-2,3-butanediol

Pinanediol=2,3-pinanediol=2,6,6-trimethylbicyclo [3.1.1]heptane-2,3-diol

Pip=pipecolinic acid

Room temperature=25° C.±2° C.

Salt=a product obtainable by combining an acid and a base

S2238=D-Phe-Pipecolyl-Arg-p-nitroanilide

s.c.=subcutaneous

THF=tetrahydrofuran

Thr=thrombin

TRI 50b=pinacol ester of TRI 50c (a prodrug of TRI 50c).

### The Products of the Disclosure

The present disclosure is predicated on the surprising observation that certain compounds have the effect of neutralising, that is reducing or eliminating, the activity of organoboronate medicaments, particularly boropeptide medicaments. The organoboronates are discussed below in more detail under the heading “Target Compounds”.

The disclosure provides the use, for the manufacture of a medicament for therapeutically neutralising (e.g. reducing in activity) an organoboronate drug, of a compound selected from organic acids comprising an aliphatic moiety substituted by a nucleophilic group and salts and prodrugs of such acids.

Also provided is a method for neutralising an organoboronate drug in a subject, comprising administering an effective amount of a compound selected from organic acids comprising an aliphatic moiety substituted by a nucleophilic group and salts and prodrugs of such acids.

The entire acid may be aliphatic, for example a linear aliphatic compound, though acids containing cyclic moieties, for example alicyclic moieties, are not excluded. The acid may contain up to 30 carbon atoms, for example, as in the case of compounds containing up to 24 carbon atoms; in one class of organic acids, there are at least 6 carbon atoms, e.g. at least 12 carbon atoms. In general terms, the compounds may contain 6, 7, 8, 9, 10, 11, 12, 13, 14, 15, 16, 17, 18, 19 or 20 carbon atoms, for example.

The acid is not a boron acid. As examples of the acids may be mentioned carboxylic, phosphonic, phosphinic and sulphonic acids.

The nucleophilic group may be a hydroxy, hydroperoxy or amino group, for example.

The aliphatic moiety may contain a fragment —C*(H)A- where C* is a chiral centre of (S)-configuration and A is a nucleophilic group. Also included are compounds containing a fragment —C(H)A- at the α-position to a carbon-carbon double bond, for example compounds containing a fragment —C(H)A-CH═C(R^{a})(H) where R^{a} comprises, e.g. is, an aliphatic moiety. The aliphatic moiety may be substituted or unsubstituted, for example substituted by one or more substituents (e.g. 1, 2 or 3 substituents) selected from A groups, halogen (e.g. F or Cl), alkyl (e.g. 1C to 4C, for example methyl), alkoxy (e.g. 1C to 4C, for example methoxy), haloalkyl (e.g. 1C to 4C, for example trifluoromethyl) and carboxy. Also contemplated is aryloxy, e.g. phenoxy.

The compounds substituted by a nucleophilic functional group may for example be aliphatic acids or be capable of releasing aliphatic acids in vivo; thus, they may be salts or esters of aliphatic acids. The acids may be carboxylic acids, e.g. mono- or di-carboxylic acids, but sulphonic acids, for example, are included in the scope of the disclosure. The compounds may be alkanes or alkenes containing 1, 2, 3, 4, 5, 6 or more double bonds (multiple double bonds may be conjugated or unconjugated; acids may include both conjugated and unconjugated double bonds), substituted by nucleophilic and acid moieties (other than boron acids), or of course salt or prodrug forms thereof. The acids may be substituted by a plurality of nucleophilic groups, particularly plural hydroxy groups (e.g. 2, 3, 4 5 or 6 hydroxy groups).

Where the acids contain one or more double bonds, they may be cis or trans; in one class of acids containing a fragment —C(H)A-CH═C(R^{a})(H), the double bond shown in the fragment is of trans (E) configuration and is optionally conjugated to a cis (Z) double bond within moiety R^{a}.

In one embodiment, the present disclosure provides a product for neutralising a boropeptidyl serine protease inhibitor, the product comprising a compound of the Formula (I) or a salt or prodrug thereof:

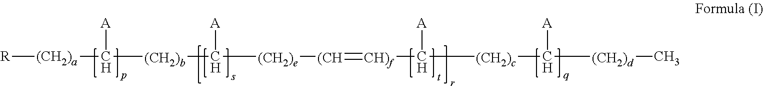

where:

R is —COOH, —P(O)(OH)₂, —HPOOH, or —SO₃H;

A is a nucleophilic group;

the sum of a and b is an integer from 4 to 9 inclusive and a or b may be 0;

the sum of c and d is an integer from 1 to 6 inclusive and c or d may be 0

e and f are independently 0 or 1, the value of each instance of e and f being independent of each other instance, if any;

r is an integer from 1 to 6, e.g. is 2, 3, 4 or 5; and

p, q, s and t are independently 0 or 1, the value of each instance of s and t being independent of the value of each other instance (if any) and there being at least one —C(H)A- moiety; and

at least one —CH₂— group within the compound may be replaced by an ether linkage —O— or an amine linkage —NH—.

Included are compounds in which there is at least one —CH═CH— moiety, i.e. in which at least one instance of f is 1. In a class of such compounds, the —CH═CH— moiety is directly bonded to a —C(H)A- moiety and, in this class, the —CH═CH— moiety may be in trans (E) configuration.

In notable classes of compounds, there is a fragment —C(H)A-CH═CH— in which A is a hydroxy group. Typically the double bond is of trans configuration. Typically, chiral centre —C(H)A- is of (S) configuration. The hydroxy group may be separated from the acid group (R in formula (I)) by 7 or 8 atoms, for example.

In one class of compounds, a+p+b is at least 5, e.g. is from 5 to 10, and the moiety R—(CH₂)ₐ-[C(H)A]ₚ—(CH₂)_{b}— is directly bonded to a moiety C(H)A-CH═CH—; the double bond may be in trans (E) configuration and in some compounds is conjugated to a double bond in cis (Z) configuration. The cis double bond is typically not further conjugated to a further double bond but the compound may contain additional double bonds, e.g. a second cis double bond separated from the first one by a single —CH₂— group. Chiral centre —C(H)A- may be of (S)-configuration. The moiety R—(CH₂)ₐ-[C(H)A]ₚ—(CH₂)_{b}— may include an ether linkage —O— or an amine linkage —N— in place of a —CH₂— group. The amine linkage may be of the formula —N(Q)-, where Q is H or 1C-6C hydrocarbyl, e.g. alkyl, optionally substituted by hydrogen, hydroxy, 1C-6C alkoxy, amino or carboxy, for example.

Where a —CH₂— group is replaced, then in a class of compounds the replacement is by an ether linkage. Sometimes a single —CH₂— group is replaced; in other compounds, two —CH₂— groups are replaced.

In particular embodiments, s and t are 0 in at least one fragment —[C(H)A]ₛ-(CH₂)ₑ—(CH═CH)_{f}-[C(H)A]ₜ—. In this case, p+q may particularly be 1. In further particular embodiments, f is 1 with e either 0 or 1 in at least one fragment —[C(H)A]ₛ-(CH₂)ₑ—(CH═CH)_{f}-[C(H)A]ₜ—. In further particular embodiments, r is 1 or 2.

In further particular embodiments, the acid of Formula (I) typically comprises from 6 to 24, particularly from 12 to 24 carbon atoms in total, in particular 14 to 20 carbon atoms, for example 18 or 20 carbon atoms. As previously described, at least one of the carbon atoms (e.g. 1, 2 or 3) may be replaced by an oxygen or nitrogen atom. Typical products of Formula (I) (whether saturated or unsaturated) may contain 1 or 2 nucleophilic groups A, for example hydroxy groups and/or hydroperoxy groups.

In these and other particular embodiments, the reversal agent is a fatty acid or a fatty acid derivative such as a fatty acid salt or fatty acid ester, for example a pharmaceutically acceptable salt such as a sodium salt, or a cholesteryl ester or alkyl ester.

Particularly R is —COOH, or of course the —COOH group may be derivatised to form a salt or prodrug, e.g. an ester.

Particularly, A is —OH, —OOH or —NH₂, more especially —OH or —OOH. In a preferred class of compounds, A is —OH.

Where any compound of the disclosure contains plural A groups, the identity of any one A group is independent from that of the other A groups. Nonetheless, in some classes of compounds, all the A groups are hydroperoxy or hydroxy; included are compounds in which there are plural A groups which are all hydroxy groups. Also, as previously indicated, there are classes of compounds containing a fragment —C(H)A-CH═CH— in which A is hydroxy.

Any one or more hydrogen atoms of the disclosed compounds may be replaced by halogen, e.g. F or Cl, of which F forms a particular example. In many classes of the illustrated formulae, those atoms which are indicated to be hydrogens are not replaced by halogen.

The neutralising acids may be of the Formula (II), or salts or prodrugs thereof:

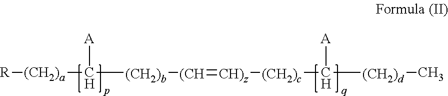

where:

R is —COOH, —P(O)(OH)₂, —HPOOH, or —SO₃H;

A is a nucleophilic group;

the sum of a and b is an integer from 4 to 9 inclusive and a or b may be 0;

the sum of c and d is an integer from 1 to 6 inclusive and c or d may be 0

z is 1 or 2; and

p and q are independently 0 or 1 and p+q is at least 1.

Included are acids in which p is 1, b is 0 and z is 1 or 2. In all acids where there is a double bond i.e. a C═C structural fragment) directly bonded to —C(H)A-, the double bond may be of trans configuration and any double bond it is conjugated to may be of cis configuration. Chiral centre —CH(A)- may be of (S)-configuration.

In further particular embodiments, the products of Formula (II) will typically comprise from 12 to 24 carbon atoms in total, in particular 14 to 20 carbon atoms, for example 18 carbon atoms. Typical products of Formula II may contain 1 or 2 nucleophilic groups A, for example hydroxy groups and/or hydroperoxy groups. In these and other particular embodiments, the product of Formula (II) is a fatty acid or a fatty acid derivative such as a fatty acid salt or fatty acid ester, for example a pharmaceutically acceptable salt such as a sodium salt, or a cholesteryl or alkyl ester.

The fatty acid or fatty acid residue of Formula (II) is singly or doubly unsaturated (that is, containing one or two C═C moieties), and in particular embodiments there is a C═C at one or two of the 8, 10, 12 and 14 positions, typically at the 10 or 12 position or both. In further particular embodiments, the nucleophilic group A may be in the α, β or γ position with respect to an unsaturated carbon, in particular in the α or β position, notably the α position. The nucleophilic group may typically be located in a 7, 8, 9, 10, 11, 12, 13 or 14 position, e.g. the 7, 9, or 11 position, such as the 9 position, for example.

Particular R and A moieties are as discussed previously.

The compounds are ideally isomerically pure, though 100% purity might not be obtainable. For example, the or each —CH(A)- chiral centre may be of (S)-configuration, excluding any impurity of (R)-configuration; in some compounds there is a single —CH(A)- chiral centre of (S) configuration, whether or not there are plural —CH(A)- chiral centres. The compound may be, for example, at least about 95% of (S)-configuration e.g. at least about 96, 97, 98, 99 or 100% of (S)-configuration, at —CH(A)- chiral centre(s) of (S)-configuration. Racemic mixtures are not excluded.

In the case of compounds having plural carbon-carbon double bonds with an α-carbon available for substitution by an A group (for example 9- or 13-HODE), the compound may be substantially mono substituted, e.g. at least about 95% mono-substituted, such as at least about 96, 97, 98, 99 or 100% mono substituted, for example.

Those compounds having just one —CH(A)- group may be isomerically pure in the sense of the group being at a single position in the molecule, excluding any impurity, for example they may contain at least about 95% of a particular isomer (e.g. 9-HODE), such as at least about 96, 97, 98, 99 or 100% of one isomer.

The disclosed compounds may be in isolated form. The compounds may be substantially pure, e.g. at least about 95% pure, for example at least about 96%, 97%, 98%, 99% or 100% pure.

Included are uses which involve compounds containing a pharmacophore (or structural fragment) of Formula (III), or a salt or prodrug thereof:

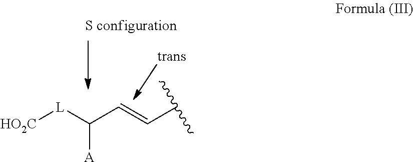

wherein:

A is a nucleophilic group; and

L is a linker containing from 5 to 10 in-chain atoms, e.g. 5, 6, 7 or 8.

A is hydroxy in a particular set of compounds. As other possible A groups may be mentioned hydroperoxy and amino.

The in-chain atoms of L may by way of example be C, O, N or S, e.g. C or O. Included is a class of compounds in which all the in-chain atoms of L are carbon. In one class of compounds there are 0, 1 or 2 in-chain atoms which are not carbon, e.g. 1 such non-carbon atom. The in-chain bonds of L are typically double or single bonds; included is a class of compounds in which all the in-chain bonds are single bonds or are all single except one double bond, usually a carbon-carbon double bond. In many compounds L is unsubstituted (all out-of-chain bonds are to hydrogen); alternatively the linker may be substituted, for example each in-chain atom may have at least one substituent or, more frequently, L contains fewer substituent groups than in-chain atoms. In one class of compounds, no in-chain atom contains more than one substituent. Included are compounds in which L has 1, 2 or 3 substituents, e.g. 1 substituent. As substituents there may be mentioned A groups, halogen (e.g. F or Cl), alkyl (e.g. 1C to 4C, for example methyl), alkoxy (e.g. 1C to 4C, for example methoxy), haloalkyl (e.g. 1C to 4C, for example trifluoromethyl) and carboxy. Included are compounds in which any substituents on L are halogen or an A group; as a particular substituent of this set may be mentioned hydroxy.

In certain compounds, L groups have 5, 6, 7 or 8 in-chain atoms, e.g. 6 or 7. Suitably all the in-chain atoms are carbon atoms. Included in the disclosure are L groups which are alkylene or alkenylene groups.

Exemplary L groups are —(CH₂)₆—, —(CH₂)₇— and —(CH₂)₃—CH═CHCH₂—; in the latter of these, the double bond may be of cis configuration.

The remainder of the molecule to which the fragment of Formula (III) is bonded may be an aliphatic group and is an open chain aliphatic group, particularly a linear aliphatic group, in one class of compounds. The aliphatic group may be completely saturated or it may be unsaturated, e.g. containing 1, 2, 3, 4 or more carbon-carbon multiple, particularly double, bonds. The aliphatic group may be a hydrocarbyl group. Included in the disclosure is a class of compounds in which the remainder of the molecule is an R^{a} moiety as described above, e.g. —(CH═CH—CH₂)ᵤ—(CH₂)ᵥ—CH₃ where u is from 1 to 5, e.g. 1, 2, 3 or 4 (for example 1, 2 or 3), and v is from 0 to 6, e.g. is 0, 1, 2, 3 or 4. To be mentioned are compounds in which u is 1, 2 or 3 and v is 1, 2, 3 or 4. Exemplary are —CH═CH—(CH₂)₄—CH₃, —CH═CH—CH₂—CH═CH—(CH₂)₄—CH₃ and —CH═CH—CH₂—CH═CH—CH₂—CH═CH—CH₂—CH₃.

In one class of compounds, the compounds have a pharmacophore of Formula (IV):

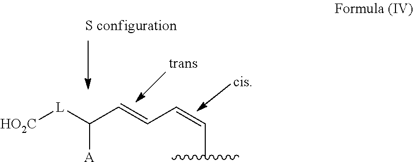

The remainder of the molecule may be an aliphatic group as discussed above, e.g. the terminal fragment —CH═CH— when taken together with the remainder of the molecule may form a remainder group as described above with reference to Formula (III).

In a sub-class, the cis (Z) double bond is not conjugated to a second trans double bond, e.g. the pharmacophore is of formula (V):

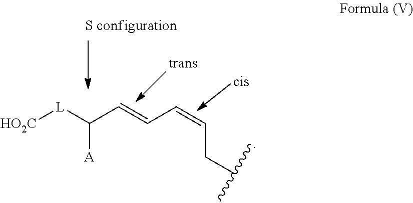

The remainder of the molecule may be an aliphatic group as discussed above, e.g. the terminal fragment —CH═CH—CH₂— when taken together with the remainder of the molecule may form a remainder group as described above with reference to Formula (III).

To be mentioned are compounds of the formula (VI):

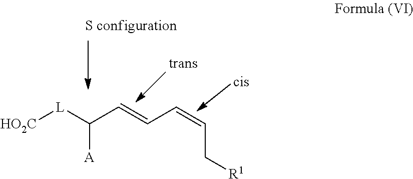

where R¹ is H or 1C-10C alkyl or 2C-10C alkenyl, optionally substituted by one or more substituents selected from A groups, halogen (e.g. F or Cl), alkoxy (e.g. 1C to 4C, for example methoxy), haloalkyl (e.g. 1C to 4C, for example trifluoromethyl) and carboxy. Where R¹ is alkyl or alkenyl, it may be branched or, more particularly, linear. Included are compounds in which any substituents on R¹ are halogen or an A group; as a particular substituent of this set may be mentioned hydroxy. In many compounds R¹ is unsubstituted; alternatively R¹ may be substituted, for example each in-chain atom may have at least one substituent or, more frequently, R¹ contains fewer substituent groups than in-chain atoms. In one class of compounds, no in-chain atom contains more than one substituent. Included are compounds in which R¹ has 1, 2 or 3 substituents, e.g. 1 substituent.

In a class of compounds, R¹ is alkyl which has 3, 4, 5, 6, 7 or 8 carbon atoms, e.g. from 4 to 7.

Where R¹ is alkenyl, it may have one or more double bonds; in the case of plural double bonds they may be conjugated or unconjugated, e.g. unconjugated. R¹ may contain both conjugated and unconjugated double bonds. Included amongst compounds having alkenyl R¹ groups are those of formula (VII):

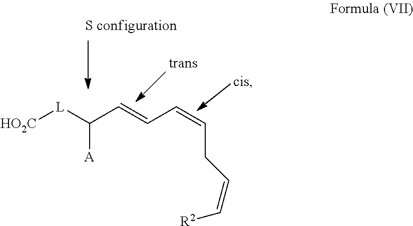

where R² is a residue of an R¹ group corresponding to the fragment R²—CH═CH—, e.g. is H or 1C to 8C alkyl or 2C to 8C alkenyl, for example 4C, 5C or 6C alkyl or alkenyl.

Particular acids of the disclosure are:

### 9(S)-HODE

The full name of this compound is 9(S)-hydroxy-10E,12Z-octadecadienoic acid and its structure is shown below.

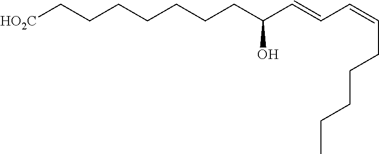

### 8(S)-HETRE

The full name of this compound is 8(S)-hydroxy-9E, 11Z, 14Z-eicosatrienoic acid and its structure is shown below.

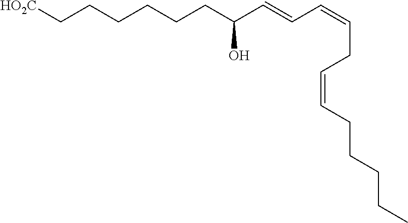

### 8(S)-HEPE

The full name of this compound is 8(S)-hydroxy-5Z, 9E, 11Z, 14Z, 17Z-eicosapentaenoic acid and its structure is shown below.

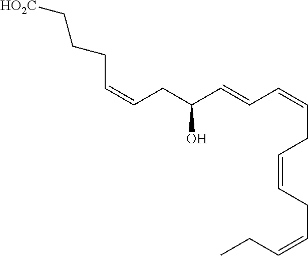

The invention includes prodrugs for the active pharmaceutical species of the invention, for example in which one or more functional groups are protected or derivatised but can be converted in vivo to the functional group, as in the case of esters of carboxylic acids convertible in vivo to the free acid, or in the case of protected amines, to the free amino group. The term “prodrug,” as used herein, represents in particular compounds which are rapidly transformed in vivo to the parent compound, for example, by hydrolysis in blood. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996), each of which is incorporated herein by reference.

Prodrugs therefore include drugs having a functional group which has been transformed into a reversible derivative thereof. Typically, such prodrugs are transformed to the active drug by hydrolysis. As examples may be mentioned the following:

| Functional Group | Reversible derivative |
|---|---|
| Carboxylic acid | Esters, including e.g. acyloxyalkyl esters, amides |
| Alcohol | Esters, including e.g. sulfates and phosphates as |
| | well as carboxylic acid esters |
| Amine | Amides, carbamates, imines, enamines, |
| Carbonyl (aldehyde, | Imines, oximes, acetals/ketals, enol esters, |
| ketone) | oxazolidines and thiazoxolidines |

The use of protecting groups is fully described in ‘Protective Groups in Organic Chemistry’, edited by J W F McOmie, Plenum Press (1973), and ‘Protective Groups in Organic Synthesis’, 2nd edition, T W Greene & P G M Wutz, Wiley-Interscience (1991).

Exemplary prodrugs are esters formed at the carboxy group of the acid. The invention is not limited as to the identity of the ester forming group: it may be a simple organic moiety, for example an alkyl (e.g. IC to 8C), alkoxyalkyl (e.g. 2C to 8C) or alkenyl group (e.g. 2C to 8C), such groups being removable by carboxyesterases and often containing 1, 2, 3 or 4 carbon atoms. Alternatively, the esters may be glycerols, e.g. triacylglycerols. Also included are esters of terpenes (notably steroids such s, for example, steroids) which have at least one hydroxy group, e.g. cholesterol. As examples may be mentioned the cholesteryl esters of 9(S)-HODE, 8(S)—HETRE and 8(S)-HEPE, the former of which has the following structure:

### 9(S)-HODE Cholesteryl Ester

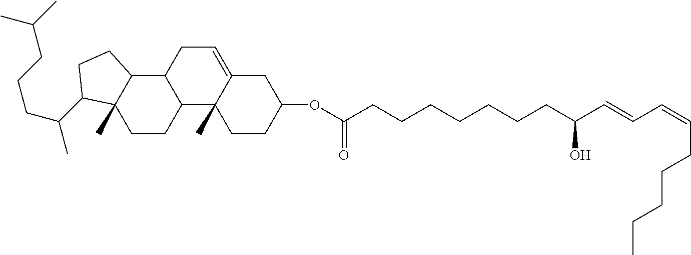

Thus, it will be appreciated by those skilled in the art that, although protected derivatives of disclosed compounds may not possess pharmacological activity as such, they may be administered, for example parenterally (notably intravenously) or orally, and thereafter metabolised in the body to form compounds which are pharmacologically active. Such derivatives and their salts are therefore examples of “prodrugs”. All prodrugs of the described acids are included within the scope of the invention.

The pharmaceutically acceptable salts of the present invention can be synthesized from the parent compound (active compound or prodrug) by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, and in Stahl et al (Eds), “Handbook of Pharmaceutical Salts Properties Selection and Use”, Wiley-VCH, 2002.

The invention thus includes pharmaceutically-acceptable salts of the disclosed compounds and their covalent prodrug molecules wherein the parent compound is modified by making acid or base salts thereof, for example the conventional non-toxic salts or the quaternary ammonium salts which are formed, e.g., from inorganic or organic acids or bases. Examples of base addition salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

The disclosed compounds (e.g. acid, salt or prodrug), may be in the form of a solvate or hydrate thereof.

It is contemplated that a plurality of the disclosed compounds may be used together and, as such, the present invention includes combinations of one or more of the compounds as disclosed herein. For example the present invention includes combinations of one or more of 9(S)-HODE, 8(S)—HETRE and 8(S)-HEPE. More usually, a single active ingredient is included in a pharmaceutical formulation.

### Target Compounds

The products of the disclosure are useful for reducing or substantially destroying the clinically significant activity of a biologically active boronate species and particularly of organoboronate drugs. Such reduction of substantial destruction of clinically significant activity of drugs are convenience referred to herein as “neutralisation”. In particular, the disclosed products find application in neutralising aminoboronates or peptidoboronates as described in more detail below.

Typically, the boronate group (—B(OH)₂ or a salt or prodrug form thereof) of the target compound is bonded to an aliphatic carbon atom and normally to an sp³ carbon atom. The target compound may for example be any boronic acid drug mentioned under the heading “BACKGROUND” or in any document referred to under that heading, e.g. it may be TRI 50c or Velcade®. It may be a boronic acid described in WO 01/02424. Particular boronic acid drugs are peptide boronic acids, including those having a C-terminal residue which is of an α-aminoboronic acid having an alkyl or alkoxyalkyl side chain. An exemplary C-terminal residue is of Boro-3-methoxypropylglycine, as for example in the case that the drug comprises a boropeptide which includes the sequence Pro-Mpg-B(OH)₂, for example as part of the larger sequence Phe-Pro-Mpg-B(OH)₂, whether administered as the free acid, a salt or a prodrug. In this paragraph, reference to a boronic acid described in the prior art includes reference to the free acids and salts of boronate esters described in the prior art. It may be any other boronic acid drug.

The target compounds for neutralisation by the products of the invention, therefore, are organoboronates and especially boropeptides. Organoboronates (e.g. peptide boronates) can exist in different forms, such as acids, esters and tautomers, for example, and the target compounds of the present invention includes all variant forms of the compounds. Whilst pharmaceutically useful boronic acids may be administered as the free acid or anhydride, they may also be administered in other forms, e.g. as salts or as esters or other prodrugs.

Thus, many organoboronates include basic groups and may therefore be administered in the form of acid addition salts. Exemplary acids include HBr, HCl and HSO₂CH₃. Alternatively the organoboronates may be administered in the form of base addition salts thereof as described in WO 2004/022072, U.S. Ser. No. 10/659,178 and EP-A-1396270; WO 2004/022071, U.S. Ser. No. 10/659,179 and EP-A-1396269; and also in WO 2004/022070, U.S. Ser. No. 10/658,971 and EP-A-1400245. Salts of alkali metal and alkaline earth metals, e.g. sodium and calcium, are representative of base addition salts as well as salts of organic bases, e.g. N-methyl-D-glucamine. The target organoboronate drugs may be administered as esters, notably esters of diols; exemplary diols in particular are sugars, for example mannitol, as described in WO 02/059131 and U.S. Pat. No. 6,699,835.

Further, there is a debate in the literature as to whether boronates in aqueous solution form the ‘trigonal’ B(OH)₂ or ‘tetrahedral’ B(OH)⁻₃ boron species, and representations of trigonal B(OH)₂ include reference to tetrahedral as well as trigonal boron species.

The target compounds of the invention therefore include all variant forms of the substances concerned, for example any tautomer or any pharmaceutically acceptable salt, ester, acid or other variant of the substances and their tautomers as well as substances which, upon administration, are capable of providing directly or indirectly such substances or providing a species which is capable of existing in equilibrium with such a substance.

In certain embodiments the organoboronic acid is hydrophobic.

Included herein are embodiments in which the organoboronic acid comprises an aminoboronic acid linked through a peptide linkage to an organic moiety, and often a moiety comprising an amino acid (natural or unnatural) or a peptide, which organic moiety may be hydrophobic. The organic moiety can comprise an amino acid whose C-terminal carboxy group forms part of said peptide linkage. The target compound may therefore be of formula (XIII):

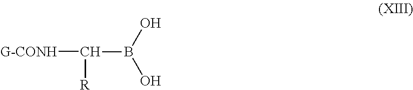

In formula (XIII), G is an organic moiety, for example comprising together with —CO— a residue of an optionally N-terminally substituted amino acid or peptide (e.g. dipeptide), a suitable N-terminal substituent being for example an X group as described below. R is a side chain of an amino acid (whether natural or unnatural). G and R may be hydrophobic. R may be an R¹ group as described below. Peptide linkages in formula (XIII) compounds are optionally and independently N-substituted, for example by a C₁-C₁₃ hydrocarbyl optionally containing in-chain oxygen or sulfur and optionally substituted by a substituent selected from halo, hydroxy and trifluoromethyl (an example of such an N-substituent is 1C to 6C alkyl).

One specific class of target compounds comprises those wherein the organoboronic acid comprises a boropeptide or boropeptidomimetic. For example, in a sub-class of these organoboronates the organoboronic acid is of the formula (VIII):

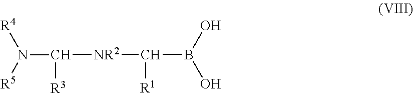

where:

R¹ is H or a non-charged side group;

R² is H or C₁-C₁₃ hydrocarbyl optionally containing in-chain oxygen or sulfur and optionally substituted by a substituent selected from halo, hydroxy and trifluoromethyl;
or R¹ and R² together form a C₁-C₁₃ moiety which in combination with N—CH forms a 4-6 membered ring and which is selected from alkylene (whether branched or linear) and alkylene containing an in-chain sulfur or linked to N—CH through a sulfur;

R³ is the same as or different from R¹ provided that no more than one of R¹ and R² is H;

R⁴ is H or a C₁-C₁₃ hydrocarbyl group optionally containing in-chain oxygen or sulfur and optionally substituted by a substituent selected from halo, hydroxy and trifluoromethyl;
or R³ and R⁴ together form a C₁-C₁₃ moiety which in combination with N—CH forms a 4-6 membered ring and which is selected from alkylene (whether branched or linear) and alkylene containing an in-chain sulfur or linked to N—CH through a sulfur; and

R⁵ is X-E- wherein E is nothing or a hydrophobic moiety selected from the group consisting of amino acids (natural or unnatural) and peptides of two or more amino acids (natural or unnatural) of which more than half are hydrophobic, in which peptides the nitrogen(s) of the peptide linkage(s) may be substituted by a C₁-C₁₃ hydrocarbyl optionally containing in-chain oxygen or sulfur and optionally substituted by a substituent selected from halo, hydroxy and trifluoromethyl (an example of such an N-substituent is 1C to 6C alkyl), and X is H or an amino-protecting group.

Said C₁-C₁₃ hydrocarbyl optionally containing in-chain oxygen or sulfur may be selected from alkyl; alkyl substituted by cycloalkyl, aryl or heterocyclyl; cycloalkyl; aryl; and/or heterocyclyl. Heterocyclyl may be heteroaryl.

R¹ may be non polar. In some embodiments, R¹ contains up to 20 carbon atoms. R¹ may have affinity for the S1 subsite of a protease.

In a preferred class of boronic acids, which are anti-thrombotic and include TRI 50c, the acid has a neutral moiety capable of binding to the thrombin S1 subsite linked to a hydrophobic moiety capable of binding to the thrombin S2 and S3 subsites. The acid may for example be of formula (III):

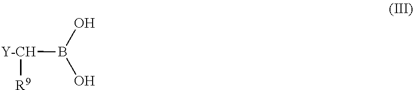

wherein

Y comprises a moiety which, together with the fragment —CH(R⁹)—B(OH)₂, has affinity for the substrate binding site of thrombin; and

R⁹ is a straight chain alkyl group interrupted by one or more ether linkages (e.g. 1 or 2) and in which the total number of oxygen and carbon atoms is 3, 4, 5 or 6 (e.g. 5) or R⁹ is —(CH₂)ₘ—W where m is 2, 3, 4 or 5 (e.g. 4) and W is —OH or halogen (F, Cl, Br or I). As examples of straight chain alkyl interrupted by one or more ether linkages (—O—) may be mentioned alkoxyalkyl (one interruption) and alkoxyalkoxyalkyl (two interruptions). R⁹ is an alkoxyalkyl group in one subset of compounds, e.g. alkoxyalkyl containing 4 carbon atoms.

The neutral aminoboronic acid residue capable of binding to the thrombin S1 subsite may be linked through a peptide linkage to a hydrophobic moiety capable of binding to the thrombin S2 and S3 subsites. As a class of such compounds may be mentioned acids of formula (IX):

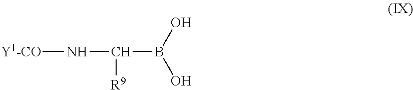

wherein

Y¹ comprises a hydrophobic moiety which, together with the aminoboronic acid residue —NHCH(R⁹)—B(OH)₂, has affinity for the substrate binding site of thrombin; and

R⁹ is as defined above.

Typically, YCO— comprises an amino acid residue (whether natural or unnatural) which binds to the S2 subsite of thrombin, the amino acid residue being N-terminally linked to a moiety which binds the S3 subsite of thrombin. Peptide linkages in the acid of formula (IX) may be substituted or unsubstituted; in one class of embodiments they are unsubstituted.

In one class of Formula (IX) acids, YCO— is an optionally N-terminally protected dipeptide residue which binds to the S3 and S2 binding sites of thrombin and the peptide linkages in the acid are optionally and independently N-substituted by a C₁-C₁₃ hydrocarbyl group optionally containing in-chain and/or in-ring nitrogen, oxygen or sulfur and optionally substituted by a substituent selected from halo, hydroxy and trifluoromethyl. The N-terminal protecting group, when present, may be a group X as defined above (other than hydrogen). Normally, the acid contains no N-substituted peptide linkages; where there is an N-substituted peptide linkage, the substituent is often 1C to 6C hydrocarbyl, e.g. saturated hydrocarbyl; the N-substituent comprises a ring in some embodiments, e.g. cycloalkyl, and may be cyclopentyl, for example. One class of acids has an N-terminal protecting group (e.g. an X group) and unsubstituted peptide linkages.

Where YCO— is a dipeptide residue (whether or not N-terminally protected), the S3-binding amino acid residue may be of R configuration and/or the S2-binding residue may of S configuration. The fragment —NHCH(R⁹)—B(OH) may of R configuration. The disclosure is not restricted to chiral centres of these conformations, however.

In one class of compounds, the side chain of P3 (S3-binding) amino acid and/or the P2 (S2-binding) amino acid is a moiety other than hydrogen selected from a group of formula A or B:

—(CO)ₐ—(CH₂)_{b}-D_{c}-(CH₂)_{d}-E (A)

—(CO)ₐ—(CH₂)_{b}-D_{c}-Cₑ(E¹)(E²)(E³) (B)

wherein

a is 0 or 1;

e is 1;

b and d are independently 0 or an integer such that (b+d) is from 0 to 4 or, as the case may be, (b+e) is from 1 to 4;

c is 0 or 1;

D is O or S;

E is H, C₁-C₆ alkyl, or a saturated or unsaturated cyclic group which normally contains up to 14 members and particularly is a 5-6 membered ring (e.g. phenyl) or an 8-14 membered fused ring system (e.g. naphthyl), which alkyl or cyclic group is optionally substituted by up to 3 groups (e.g. 1 group) independently selected from C₁-C₆ trialkylsilyl, —CN, —R¹³, —R¹²OR¹³, —R¹²COR¹³, —R¹²CO₂R¹³ and —R¹²O₂CR¹³, wherein R¹² is —(CH₂)_{f}— and R¹³ is —(CH₂)_{g}H or by a moiety whose non-hydrogen atoms consist of carbon atoms and in-ring heteroatoms and number from 5 to 14 and which contains a ring system (e.g. an aryl group) and optionally an alkyl and/or alkylene group, wherein f and g are each independently from 0 to 10, g particularly being at least 1 (although —OH may also be mentioned as a substituent), provided that (f+g) does not exceed 10, more particularly does not exceed 6 and most particularly is 1, 2, 3 or 4, and provided that there is only a single substituent if the substituent is a said moiety containing a ring system, or E is C₁-C₆ trialkylsilyl; and E¹, E² and E³ are each independently selected from —R¹⁵ and -J-R¹⁵, where J is a 5-6 membered ring and R¹⁵ is selected from C₁-C₆ trialkylsilyl, —CN, —R¹³, —R¹²OR¹³, —R¹²COR¹³, —R¹²CO₂R¹³, —R¹²O₂CR¹³, and one or two halogens (e.g. in the latter case to form a -J-R¹⁵ moiety which is dichlorophenyl), where R¹² and R¹³ are, respectively, an R¹² moiety and an R¹³ moiety as defined above (in some acids where E¹, E² and E³ contain an R¹³ group, g is 0 or 1);

in which moiety of Formula (A) or (B) any ring is carbocyclic or aromatic, or both, and any one or more hydrogen atoms bonded to a carbon atom is optionally replaced by halogen, especially F.

In certain examples, a is 0. If a is 1, c may be 0. In particular examples, (a+b+c+d) and (a+b+c+e) are no more than 4 and are more especially 1, 2 or 3. (a+b+c+d) may be 0.

Exemplary groups for E, E¹, E² and E³ include aromatic rings such as phenyl, naphthyl, pyridyl, quinolinyl and furanyl, for example; non-aromatic unsaturated rings, for example cyclohexenyl; saturated rings such as cyclohexyl, for example. E may be a fused ring system containing both aromatic and non-aromatic rings, for example fluorenyl. One class of E, E¹, E² and E³ groups are aromatic (including heteroaromatic) rings, especially 6-membered aromatic rings. In some compounds, E¹ is H whilst E² and E³ are not H; in those compounds, examples of E² and E³ groups are phenyl (substituted or unsubstituted) and C₁-C₄ alkyl, e.g. methyl.

In one class of boronic acids, E contains a substituent which is C₁-C₆ alkyl, (C₁-C₅ alkyl)carbonyl, carboxy C₁-C₅ alkyl, aryl (including heteroaryl), especially 5-membered or preferably 6-membered aryl (e.g. phenyl or pyridyl), or arylalkyl (e.g. arylmethyl or arylethyl where aryl may be heterocyclic and is preferably 6-membered).

In another class of boronic acids, E contains a substituent which is OR¹³, wherein R¹³ can be a 6-membered ring, which may be aromatic (e.g. phenyl) or is alkyl (e.g. methyl or ethyl) substituted by such a 6-membered ring.

A class of moieties of formula A or B are those in which E is a 6-membered aromatic ring optionally substituted, particularly at the 2-position or 4-position, by —R¹³ or —OR¹³.

Also to be mentioned are boronic aid thrombin inhibitors in which the P3 and/or P2 side chain comprises a cyclic group in which 1 or 2 hydrogens have been replaced by halogen, e.g. F or Cl. Further to be mentioned is a class of organoboronic acid in which the side chains of formula (A) or (B) are of the following formulae (C), (D) or (E):

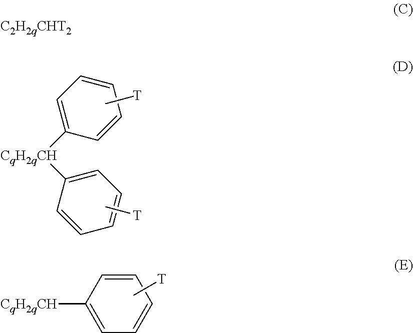

wherein q is from 0 to 5, e.g. is 0, 1 or 2, and each T is independently hydrogen, one or two halogens (e.g. F or Cl), —SiMe₃, —CN, —R¹³, —OR¹³, —COR¹³, —CO₂R¹³ or —O₂CR¹³. In some embodiments of structures (D) and (E), T is at the 4-position of the phenyl group(s) and is —R¹³, —OR¹³, —COR¹³, —CO₂R¹³ or —O₂CR¹³, and R¹³ is C₁-C₁₀ alkyl and more particularly C₁-C₆ alkyl. In one sub-class, T is —R¹³ or —OR¹³, for example in which f and g are each independently 0, 1, 2 or 3; in some side chains groups of this sub-class, T is —R¹²OR¹³ and R¹³ is H.

In one class of the moieties, the side chain is of formula (C) and each T is independently R¹³ or OR¹³ and R¹³ is C₁-C₄ alkyl. In some of these compounds, R¹³ is branched alkyl and in others it is straight chain. In some moieties, the number of carbon atoms is from 1 to 4.

In many dipeptide fragments YCO— (which dipeptides may be N-terminally protected or not), the P3 amino acid has a side chain of formula (A) or (B) as described above and the P2 residue is of an imino acid.

The target compounds may therefore be organoboronic acids which are thrombin inhibitors, particularly selective thrombin inhibitors, having a neutral P1 (S1-binding) moiety. For more information about moieties which bind to the S3, S2 and S1 sites of thrombin, see for example Tapparelli C et al, Trends Pharmacol. Sci. 14: 366-376, 1993; Sanderson P et al, Current Medicinal Chemistry, 5: 289-304, 1998; Rewinkel J et al, Current Pharmaceutical Design, 5:1043-1075, 1999; and Coburn C Exp. Opin. Ther. Patents 11(5): 721-738, 2001. The thrombin inhibitory compounds are not limited to those having S3, S2 and S1 affinity groups described in the publications listed in the preceding sentence.

The boronic acids may have a Ki for thrombin of about 100 nM or less, e.g. about 20 nM or less.

A subset of the Formula (IX) acids comprises the acids of Formula (X):

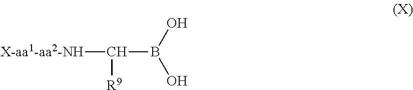

X is a moiety bonded to the N-terminal amino group and may be H to form NH₂. The identity of X is not critical but may be a particular X moiety described above. In one example there may be mentioned benzyloxycarbonyl.

In certain examples X is R⁶—(CH₂)ₚ—C(O)—, R⁶—(CH₂)ₚ—S(O)₂—, R⁶—(CH₂)ₚ—NH—C(O)— or R⁶—(CH₂)ₚ—O—C(O)— wherein p is 0, 1, 2, 3, 4, 5 or 6 (of which 0 and 1 are preferred) and R⁶ is H or a 5 to 13-membered cyclic group optionally substituted by 1, 2 or 3 substituents selected from halogen, amino, nitro, hydroxy, a C₅-C₆ cyclic group, C₁-C₄ alkyl and C₁-C₄ alkyl containing, and/or linked to the 5 to 13-membered cyclic group through, an in-chain O, the aforesaid alkyl groups optionally being substituted by a substituent selected from halogen, amino, nitro, hydroxy and a C₅-C₆ cyclic group. More particularly X is R⁶—(CH₂)ₚ—C(O)— or R⁶—(CH₂)ₚ—O—C(O)— and p is 0 or 1. Said 5 to 13-membered cyclic group is often aromatic or heteroaromatic, for example is a 6-membered aromatic or heteroaromatic group. In many cases, the group is not substituted.

Exemplary X groups are (2-pyrazine) carbonyl, (2-pyrazine) sulfonyl and particularly benzyloxycarbonyl.

aa¹ is an amino acid residue having a hydrocarbyl side chain containing no more than 20 carbon atoms (e.g. up to 15 and optionally up to 13 C atoms) and comprising at least one cyclic group having up to 13 carbon atoms. In certain examples, the cyclic group(s) of aa¹ have/has 5 or 6 ring members. For instance, the cyclic group(s) of aa¹ may be aryl groups, particularly phenyl. Typically, there are one or two cyclic groups in the aa¹ side chain. Certain side chains comprise, or consist of, methyl substituted by one or two 5- or 6-membered rings.

More particularly, aa¹ is Phe, Dpa or a wholly or partially hydrogenated analogue thereof. The wholly hydrogenated analogues are Cha and Dcha.

aa² is an imino acid residue having from 4 to 6 ring members. Alternatively, aa² is Gly N-substituted by a C₃-C₁₃ hydrocarbyl group, e.g. a C₃-C₈ hydrocarbyl group comprising a C₃-C₆ hydrocarbyl ring; the hydrocarbyl group may be saturated, for example exemplary N-substituents are cyclopropyl, cyclobutyl, cyclopentyl and cyclohexyl. As a hydrocarbyl group containing one or more unsaturated bonds may be mentioned phenyl and methyl or ethyl substituted by phenyl, e.g. 2-phenylethyl, as well as β,β-dialkylphenylethyl.

An exemplary class of products comprises those in which aa² is a residue of an imino acid of formula (XI)

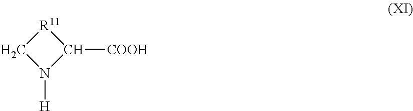

where R¹¹ is —CH₂—, CH₂—CH₂—, —S—CH₂— or —CH₂—CH₂—CH₂—, which group when the ring is 5 or 6-membered is optionally substituted at one or more —CH₂— groups by from 1 to 3 C₁-C₃ alkyl groups, for example to form the R¹¹ group —S—C(CH₃)₂—. Of these imino acids, azetidine-2-carboxylic acid, especially (s)-azetidine-2-carboxylic acid, and more particularly proline are illustrative.

It will be appreciated from the above that a particular class of organoboronates consists of those in which aa¹-aa² is Phe-Pro. In another preferred class, aa¹-aa² is Dpa-Pro. In other products, aa¹-aa² is Cha-Pro or Dcha-Pro. Of course, also included are corresponding product classes in which Pro is replaced by (s)-azetidine-2-carboxylic acid.

R⁹ is as defined previously and may be a moiety R¹ of the formula —(CH₂)ₛ-Z. Integer s is 2, 3 or 4 and W is —OH, —OMe, —OEt or halogen (F, Cl, I or, preferably, Br). Particularly illustrative Z groups are —OMe and —OEt, especially —OMe. In certain examples s is 3 for all Z groups and, indeed, for all compounds of the disclosure. Particular R¹ groups are 2-bromoethyl, 2-chloroethyl, 2-methoxyethyl, 4-bromobutyl, 4-chlorobutyl, 4-methoxybutyl and, especially, 3-bromopropyl, 3-chloropropyl and 3-methoxypropyl. Most preferably, R¹ is 3-methoxypropyl. 2-Ethoxyethyl is another preferred R¹ group.

A specific class of target compounds comprises boropeptides having the amino acid sequence Phe-Pro-BoroMpg, particularly (R)-Phe-(S)-Pro-(R)-BoroMpg. Thus, there may be mentioned acids of the formula X-Phe-Pro-Mpg-B(OH)₂, especially Cbz-Phe-Pro-Mpg-B(OH)₂; also included are analogues of these compounds in which Mpg is replaced by a residue with another of the R¹ groups and/or Phe is replaced by Dpa or another aa¹ residue.

The aa¹ moiety is preferably of R configuration. The aa² moiety is preferably of (S)-configuration. Particularly preferred target compounds of formula (III) have aa¹ of (R)-configuration and aa² of (S)-configuration. The chiral centre —NH—CH(R¹)—B— is preferably of (R)-configuration. It is considered that commercial formulations will have the chiral centres in (R,S,R) arrangement, as for example in the case of Cbz-Phe-Pro-BoroMpg-OH:

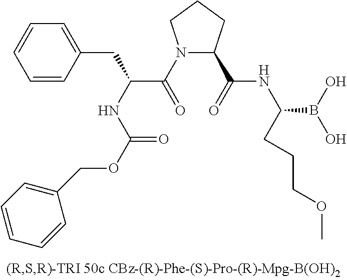

The target boronic acids may of course be administered in any form which results in release of the free acid or a corresponding boronate anion, e.g. as salts or prodrugs thereof. All the boronic acids described herein may therefore be administered in the form of prodrugs, or as the reaction product (salt) of combining the boronic acid or a prodrug thereof with a pharmaceutically acceptable acid or base, and the disclosed reversal agents may be used following administration of a boronic acid drug in free form or in salt or prodrug form.

As suitable prodrugs may be mentioned esters, e.g. with a residue of an alkanol, e.g. a C₁-C₄ alkanol such as methanol or ethanol, for example. It may be an ester of a diol.

The identity of the diol is not critical. As suitable diols may be mentioned aliphatic and aromatic compounds having hydroxy groups that are substituted on adjacent carbon atoms or on carbon atoms substituted by another carbon. That is to say, suitable diols include compounds having at least two hydroxy groups separated by at least two connecting carbon atoms in a chain or ring. One class of diols comprises hydrocarbons substituted by exactly two hydroxy groups. One such diol is pinacol and another is pinanediol and a third is diethanolamine; there may also be mentioned neopentylglycol, 1,2-ethanediol, 1,2-propanediol, 1,3-propanediol, 2,3-butanediol, 1,2-diisopropylethanediol, 5,6-decanediol and 1,2-dicyclohexylethanediol.

The prodrug may be a sugar derivative as described in WO 02/059131 (see above). Thus, the boronate group may be esterified with a sugar such as a monosaccharide or disaccharide, for example. The sugar may be a reduced sugar, e.g. mannitol or sorbitol; it may be any individual sugar or class of sugars taught in WO 02/059131. The boronic acid, sugar (or other diol) and water may be combined and then lyophilised, for example as taught in WO 02/059131.

As salts, there may be mentioned:

1. Alkali metal salts;
2. Divalent, e.g. alkaline earth metal, salts;
3. Group III metals;
4. Salts of strongly basic organic nitrogen-containing compounds, including:
4A. Salts of guanidines and their analogues;
4B. Salts of strongly basic amine, examples of which include (i) aminosugars and (ii) other amines.

Of the above salts, particularly illustrative are alkali metals, especially Na and Li, and alkaline earth metals, especially magnesium and calcium. Also illustrative are aminosugars. The term “salt” herein does not imply any particular structure at the molecular level but refers merely to a product formed by contacting together an acid and a base.

Specific salts are of the acid boronate though in practice the acid salts may contain a very small proportion of the doubly deprotonated boronate. The term “acid boronate” refers to trigonal —B(OH)₂ groups in which one of the B—OH groups is deprotonated as well as to corresponding tetrahedral groups in equilibrium therewith. Acid boronates have a stoichiometry consistent with single deprotonation.

Suitable organic bases include those with a pKb of 7 or more, e.g. 7.5 or more, for example in the region of 8 or more. Bases which are less lipophilic [e.g. have at least one polar functional group (e.g. 1, 2 or 3 such groups) for example hydroxy] are favoured; thus aminosugars are one favoured class of base, for example N-methyl-D-glucamine. Other organic bases to be mentioned are arginine and lysine.

Certain of the disclosed reversal agents contain a moiety —C(H)A- of (S)-configuration and are useful for neutralising the activity of boronic acids in which the boronate group is attached to a carbon atom forming a chiral centre of (R)-configuration. Particularly to be mentioned is a sub-class of reversal agents containing a fragment —C(H)A-CH═CH— in which moiety —C(H)A- of (S)-configuration and the double bond is of trans configuration. Accordingly, such neutralising agents are contemplated for use with boronic acid species corresponding to formulae (III) or (IX) in which fragment —C(H)R⁹— constitutes a chiral centre of (R)-configuration; particularly to be noted as neutralising agents in this context are compounds having a pharmacophore of formula (III) or (IV).

It is also worth mentioning that target compounds of formula (X) of (R,S,R) configuration (e.g. TRI 50c of that configuration, whether administered as the free acid or anhydride, anhydride salts, or as a salt or prodrug) are in particular susceptible to neutralising agents having a pharmacophore of formula (IV), (V) or (VI), notably linear aliphatic fatty acids which contain such a pharmacophore.

### Use of the Products of the Disclosure

The products of the disclosure may be used as reversal agents (also known as antidotes) for organoboronate drugs, e.g. one described herein under the heading “Target Compounds”. They may therefore be administered when an organoboronate drug has produced unwanted side effects, e.g. bleeding after administration of an anticoagulant. The products of the disclosure may also be used in any circumstances where an organoboronate compound has been ingested or absorbed and is causing toxicity.

The disclosure includes a method of preparing to supply a first pharmaceutical composition for the treatment of unwanted coagulation (e.g. thrombosis) by prophylaxis or therapy and, if required, a second pharmaceutical composition to inhibit the action of the first composition, comprising stocking a pharmaceutical composition comprising a target compound as hereinbefore described and stocking a pharmaceutical formulation or medicament comprising a product of the present disclosure.

According to a yet further aspect of the disclosure there is provided a method of providing a medicament pair, the pair comprising a first medicament for the treatment of unwanted coagulation (e.g. thrombosis) by prophylaxis or therapy and a second medicament to inhibit the action of the first medicament in the event of undue bleeding, wherein the first medicament comprises a target compound as hereinbefore described and the second medicament comprises a pharmaceutical composition or medicament according to the present disclosure.

According to a yet further aspect of the disclosure there is provided a method for treating unwanted coagulation (e.g. thrombosis) by prophylaxis or therapy, or inhibiting thrombosis in the treatment of disease by prophylaxis or therapy, using an anticoagulant which results in inappropriate bleeding and then inhibiting the action of said anticoagulant, wherein a therapeutically effective amount of an anticoagulant composition comprising a target compound as hereinbefore described is administered to a patient in need thereof, or to an extracorporeal blood circuit of a patient, to treat coagulation or inhibit coagulation in the treatment of disease (including in treatment by surgery), and, after the inappropriate bleeding, a therapeutically effective amount of a product the disclosure is administered to a patient to inhibit the anticoagulant.

It may be mentioned by way of non-limiting example that the described active products and pharmaceutical formulations may administered orally. More typically, they may be administered intravenously.

According to a yet further aspect of the disclosure there is provided use, for the manufacture of a medicament pair comprising a first medicament for treating unwanted coagulation (e.g. thrombosis) by prophylaxis or therapy and a second medicament for, if required, stopping or reducing the anticoagulant treatment, of a target compound as herein before described, for the manufacture of the first medicament and a product of the present disclosure for the manufacture of the second medicament.

According to a yet further aspect of the disclosure there is provided use, for the manufacture of a medicament pair comprising a first medicament for treating unwanted coagulation (e.g. thrombosis) by prophylaxis or therapy and a second medicament for, if required, stopping or reducing undue or inappropriate bleeding caused by the first medicament, of a target compound as herein before described for the manufacture of the first medicament and a product according to the present disclosure for the manufacture of the second medicament.

According to a yet further aspect of the disclosure there is provided a method of preparing for the administration to a patient or an extracorporeal blood circuit of a first pharmaceutical composition for the treatment of unwanted coagulation (e.g. thrombosis) by prophylaxis or therapy and, if required, a second pharmaceutical composition for reacting with the active agent of the first composition to inactivate molecules thereof, comprising supplying a pharmaceutical composition comprising a target compound, as hereinbefore described, and supplying a pharmaceutical composition or a medicament according to the present disclosure.

Particularly, either or both of the first and second pharmaceutical compositions are administered orally and/or intravenously.

Preferred target compounds are thrombin inhibitors. They are therefore useful for inhibiting thrombin. There are therefore provided compounds which have potential for controlling haemostasis and especially for inhibiting coagulation, for example in the treatment or prevention of secondary events after myocardial infarction. The medical use of the compounds may be prophylactic (including to treat thrombosis as well as to prevent occurrence of thrombosis) as well as therapeutic (including to prevent re-occurrence of thrombosis or secondary thrombotic events).

The anticoagulant target compounds may be employed when an anti-thrombogenic agent is needed. Further, it has been found that anti-thrombotic target compounds, including those of boronic acids of Formula (IX), are beneficial in that the class is useful for treating arterial thrombosis by therapy or prophylaxis. The target compounds are thus indicated in the treatment or prophylaxis of thrombosis and hypercoagulability in blood and tissues of animals including man. The term “thrombosis” includes inter alia atrophic thrombosis, arterial thrombosis, cardiac thrombosis, coronary thrombosis, creeping thrombosis, infective thrombosis, mesenteric thrombosis, placental thrombosis, propagating thrombosis, traumatic thrombosis and venous thrombosis.

It is known that hypercoagulability may lead to thromboembolic diseases.

Particular uses which may be mentioned for boronic acid inhibitors of coagulation serine proteases, e.g. thrombin inhibitors, include the therapeutic and/or prophylactic treatment of venous thrombosis and pulmonary embolism. Preferred indications envisaged for the described thrombin inhibitory boronic acids (notably the salts of TRI 50c) include:
Prevention of venous thromboembolic events (e.g. deep vein thrombosis and/or pulmonary embolism). Examples include patients undergoing orthopaedic surgery such as total hip replacement, total knee replacement, major hip or knee surgery; patients undergoing general surgery at high risk for thrombosis, such as abdominal or pelvic surgery for cancer; and in patients bedridden for more than 3 days and with acute cardiac failure, acute respiratory failure, infection.
Prevention of thrombosis in the haemodialysis circuit in patients, in patients with end stage renal disease.
Prevention of cardiovascular events (death, myocardial infarction, etc) in patients with end stage renal disease, whether or not requiring haemodialysis sessions.
Prevention of venous thrombo-embolic events in patients receiving chemotherapy through an indwelling catheter.
Prevention of thromboembolic events in patients undergoing lower limb arterial reconstructive procedures (bypass, endarteriectomy, transluminal angioplasty, etc).
Treatment of venous thromboembolic events.
Prevention of cardiovascular events in acute coronary syndromes (e.g. unstable angina, non Q wave myocardial ischaemia/infarction), in combination with another cardiovascular agent, for example aspirin (acetylsalicylic acid; aspirin is a registered trade mark in Germany), thrombolytics (see below for examples), antiplatelet agents (see below for examples).
Treatment of patients with acute myocardial infarction in combination with acetylsalicylic acid, thrombolytics (see below for examples).
Prevention of unwanted bleeding during coronary artery bypass graft procedures.

### Administration and Pharmaceutical Formulations

The disclosed compounds may be administered to a host, for example, when an organoboronate drug is resulting in undesired effects which it is wished to stop or reduce. In the case of larger animals, such as humans, the compounds may be administered alone or in combination with pharmaceutically acceptable diluents, excipients or carriers. The term “pharmaceutically acceptable” includes acceptability for both human and veterinary purposes, of which acceptability for human pharmaceutical use is preferred.

The compounds of the disclosure may be combined and/or co-administered with another medicament. For example, they may be combined and/or co-administered with a procoagulant when the target boronic acid drug is an anticoagulant.

Actual dosage levels of active ingredients in the pharmaceutical compositions of this disclosure may be varied so as to obtain an amount of the active compound(s) that is effective to achieve the desired therapeutic response for a particular patient, composition, and mode of administration (referred to herein as a “therapeutically effective amount”). The selected dosage level will depend upon the activity of the particular compound, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

According to a further aspect there is provided a parenteral formulation including a compound as described herein. The formulation may consist of the compound alone or it may contain additional components, in particular the compound may be in combination with a pharmaceutically acceptable diluent, excipient or carrier, for example a tonicity agent for the purpose of making the formulation substantially isotonic with the body of the subject to receive the formulation, e.g. with human plasma. The formulation may be in ready-to-use form or in a form requiring reconstitution prior to administration. More particularly, the parenteral formulation may be an intravenous formulation.

A particular embodiment resides in intravenous formulations, whether in liquid ready-to-use form or in solid form for reconstitution, or otherwise, comprising an acid having a pharmacophore of formula III, IV, V, or VI or a salt or prodrug thereof.

It is currently contemplated that, in the case of parenteral administration, for example i.v. administration, the compounds might for instance be administered to a 70 kg adult patient, usually as a bolus, in an amount of at least about 200 mg, e.g. about 500 mg or more and often in an amount of about 1 g or more, calculated as 9-HODE. It is envisaged that the dosage is unlikely to exceed about 3 g and more probably will not exceed about 2 g, e.g. will be no more than about 1.5 g; in some cases, the maximum dosage may be about 1.25 g, again calculated as 9-HODE. In accordance with normal medical practice, the dosage may be varied for patients of different weights, although it is anticipated that small variations from 70 kg will be disregarded.

Included in the disclosure are unit dosage forms containing a number of moles of the acid equivalent to those contained in the 9-HODE dosages mentioned in the previous paragraph, e.g. equivalent to from about 500 mg to about 1500 mg 9-HODE, for example equivalent to about 1 g of 9HODE.

Parenteral preparations can be administered by one or more routes, such as intravenous, subcutaneous, intradermal and infusion; a particular example is intravenous. A formulation disclosed herein may be administered using a syringe, injector, plunger for solid formulations, pump, or any other device recognized in the art for parenteral administration.

Liquid dosage forms for parenteral administration may include solutions, suspensions, liposome formulations, or emulsions in oily or aqueous vehicles. In addition to the active compounds, the liquid dosage forms may contain other compounds. Tonicity agents (for the purpose of making the formulations substantially isotonic with the subject's body, e.g. with human plasma) such as, for instance, sodium chloride, sodium sulfate, dextrose, mannitol and/or glycerol may be optionally added to the parenteral formulation. A pharmaceutically acceptable buffer may be added to control pH. Thickening or viscosity agents, for instance well known cellulose derivatives (e.g. methylcellulose, carboxymethylcellulose, hydroxyethylcellulose and hydroxypropylmethylcellulose), gelatin and/or acacia, may optionally be added to the parenteral formulation.

Solid dosage forms for parenteral administration may encompass solid and semi-solid forms and may include pellets, powders, granules, patches, and gels. In such solid dosage forms, the active compound is typically mixed with at least one inert, pharmaceutically acceptable excipient or carrier. The disclosed compounds may be presented as solids in finely divided solid form, for example they may be milled or micronised.

The formulations may also include antioxidants and/or preservatives. As antioxidants may be mentioned thiol derivatives (e.g. thioglycerol, cysteine, acetylcysteine, cystine, dithioerythreitol, dithiothreitol, glutathione), tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride. The parenteral formulations may be prepared as large volume parenterals (LVPs), e.g. larger than 100 ml, more particularly about 250 ml, of a liquid formulation of the active compound. Examples of LVPs are infusion bags. The parenteral formulations may alternatively be prepared as small volume parenterals (SVPs), e.g. about 100 ml or less of a liquid formulation of the active compound. Examples of SVPs are vials with solution, vials for reconstitution, prefilled syringes for injection and dual chamber syringe devices.

The formulations of the disclosure include those in which the active compound is 9(S)-HODE, 8(S)-HETRE or 8(S)-HEPE. The compounds mentioned in this paragraph, or their salts or prodrugs, may be administered as solutions or suspensions in water, typically containing one or more additives, for example isotonicity agent(s) and/or antioxidant(s). A way to store the compounds is in solid form, for example as dry powder, and to make them up into solutions for administration prior to administration. Alternatively, the compounds may be stored as liquid formulations ready for use.

One class of formulations disclosed herein is intravenous formulations. For intravenously administered formulations, the active compound or compounds can be present at varying concentrations, with a carrier acceptable for parenteral preparations making up the remainder. Particularly, the carrier is water, particularly pyrogen free water, or is aqueous based. Particularly, the carrier for such parenteral preparations is an aqueous solution comprising a tonicity agent, for example a sodium chloride solution.

By “aqueous based” is meant that formulation comprises a solvent which consists of water or of water and water-miscible organic solvent or solvents; as well as containing a compound of disclosure in dissolved form, the solvent may have dissolved therein one or more other substances, for example an antioxidant and/or an isotonicity agent. As organic cosolvents may be mentioned those water-miscible solvents commonly used in the art, for example propyleneglycol, polyethyleneglycol 300, polyethyleneglycol 400 and ethanol. Preferably, organic co-solvents are only used in cases where the active agent is not sufficiently soluble in water for a therapeutically effective amount to be provided in a single dosage form. As previously indicated, the disclosure includes formulations of alkali metal salts of the disclosed acids having a solvent which consists of water.

The solubility of the active compound in the present formulations may be such that the turbidity of the formulation is lower than 50 NTU, e.g. lower than 20 NTU such as lower than 10 NTU.

It is desirable that parenteral formulations are administered at or near physiological pH. It is believed that administration in a formulation at a high pH (i.e., greater than 8) or at a low pH (i.e., less than 5) is undesirable. In particular, it is contemplated that the formulations would be administered at a pH of between 6.0 and 7.0 such as a pH of 6.5.

The parenteral formulation may be purged of air when being packaged. The parenteral formulation may be packaged in a sterile container, e.g. vial, as a solution, suspension, gel, emulsion, solid or a powder. Such formulations may be stored either in ready-to-use form or in a form requiring reconstitution prior to administration.

Parenteral formulations according to the disclosure may be packaged in containers. Containers may be chosen which are made of material which is non-reactive or substantially non-reactive with the parenteral formulation. Glass containers or plastics containers, e.g. plastics infusion bags, may be used. A concern of container systems is the protection they afford a solution against UV degradation. If desired, amber glass employing iron oxide or an opaque cover fitted over the container may afford the appropriate UV protection.

Plastics containers such as plastics infusion bags are advantageous in that they are relatively light weight and non-breakable and thus more easily stored. This is particularly the case for Large Volume parenterals.

The intravenous preparations may be prepared by combining the active compound or compounds with the carrier. After the formulation is mixed, it may be sterilized, for example using known methods. Once the formulation has been sterilized, it is ready to be administered or packaged, particularly in dark packaging (e.g. bottles or plastics packaging), for storage. It is envisaged, however, that the disclosed compounds might not be stored in solution but as dry solids, particularly a finely divided form such as, for example, a lyophilisate, in order to prolong shelf life; this would of course apply to other parenteral formulations, not only intravenous ones.

The intravenous preparations may take the form of large volume parenterals or of small volume parenterals, as described above.

In a specific embodiment, the present disclosure is directed to products, particularly kits, for producing a single-dose administration unit. The products (kits) may each contain both a first container having the active compound (optionally combined with additives, for example anti-oxidant, preservative and, in some instances, tonicity agent) and a second container having the carrier/diluent (for example water, optionally containing one or more additives, for example tonicity agent). As examples of such products may be mentioned single and multi-chambered (e.g. dual-chamber) pre-filled syringes; exemplary pre-filled syringes are available from Vetter GmbH, Ravensburg, Germany. Such dual chamber syringes or binary syringes will have in one chamber a dry preparation including or consisting of the active compound and in another chamber a suitable carrier or diluent such as described herein. The two chambers are joined in such a way that the solid and the liquid mix to form the final solution.

The active compound and the carrier are typically combined, for example in a mixer. After the formulation is mixed, it is preferably sterilized, such as with U.V. radiation. Once the formulation has been sterilized, it is ready to be injected or packaged for storage. It is envisaged, however, that the disclosed compounds will not be stored in liquid formulation but as dry solids, in order to prolong shelf life.

It will be understood from the aforegoing that there are provided pharmaceutical products comprising an alkali metal salt, particularly sodium salt, of a disclosed hydroxy fatty acid in dry fine particle form, suitable for reconstitution into an aqueous read-to-use parenteral formulation. The alkali metal salt is suitably an acid salt. The alkali metal salt may be in a small volume parenteral unit dosage form. The alkali metal salt may be presented in a form, e.g. dry powder form, suitable for reconstituting as a large volume parenteral. One example is a sodium salt of 9(S)-HODE, 8(S)-HETRE or 8(S)-HEPE in dry powder form for reconstitution as a liquid intravenous formulation (solution) containing a tonicity agent, particularly sodium chloride. The dry powder form of a salt used in a parenteral formulation may be a lyophilisate. The reconstituted solution may be administered by injection or infusion.

Also provided are liquid formulations, e.g. solutions, comprising a liquid vehicle (typically water) and species which will result in in vivo hydroxy fatty acid upon administration of the formulation. The species may comprise a hydroxy fatty acid, the deprotonated acid or a protected form of the fatty acid (whether or not in ionised form).

Further, the compounds of the invention may be used in combination with the target compounds. The compounds of the invention may be used in this way when they are formulated to have a predetermined release time. In this way, the period of activity of the target compound may be predetermined in that, just prior to expiration of the predetermined period of activity, a compound of the present invention is released in the patient and the activity of the target compound is neutralised.

### EXAMPLES

### Example 1

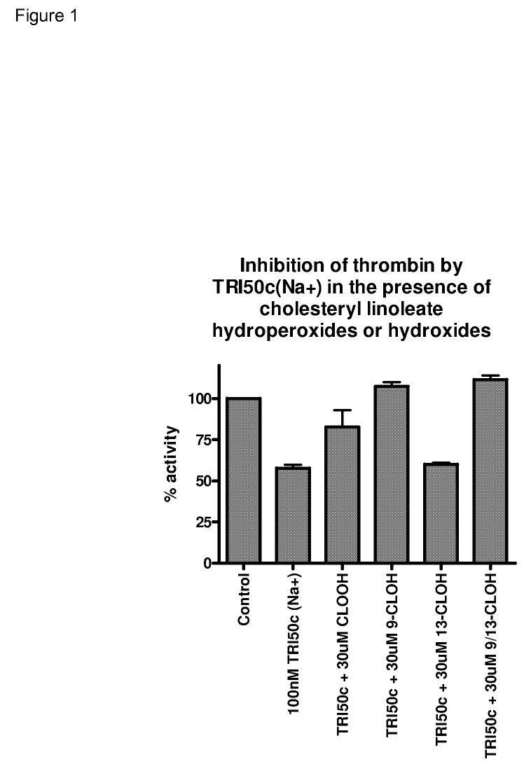

### Assays of FIGS. 1 to 3

### Materials

Peptide boronate inhibitors were synthesised by Trigen Limited. Thrombin was obtained from Haematologic Technologies Inc. Chromogenic substrate was obtained from Quadratech. Linoleic acid analogues were obtained from Cayman Chemicals USA. Buffer reagents were obtained from Merck.

### Thrombin Amidolytic Assay

Peptide boronate inhibitor of thrombin (TRI 50c, 100 nM) was incubated with thrombin (33.3 ng/ml) in assay buffer (100 mM sodium phosphate, 200 mM NaCl, 0.5% PEG 6000 and 0.02% sodium azide, pH 7.5) for 5 minutes at 37° C. Test compounds were then added and incubated for various amounts of time prior to the addition of S2238 chromogenic substrate (5 uM). The reaction was followed at 405 nM and 37° C. using a Thermomax kinetic plate reader (Molecular Devices Corporation). Results of the assay are expressed as percentage activities of thrombin relative to that seen in the absence of inhibitor or antidote.

Referring in particular to FIG. 1, it will be seen that certain fatty acid esters are effective in reducing the thrombin inhibiting activity of the peptide boronate inhibitor TRI 50c (as defined above). FIG. 1 shows the assay results for the TRI 50c-neutralising activity of the materials:

CLOOH—cholesterol hydroperoxylinoleic acid ester;

9-CLOH —9-hydroxy cholesterol linoleic acid ester;

13-CLOH —13- hydroxy cholesterol linoleic acid ester;

9/13-CLOH —9,13- hydroxy cholesterol linoleic acid ester.

The results in FIG. 1 show that, as compared with the control (no TRI 50c present), the addition of 100 nM TRI 50c reduces the thrombin activity to approximately 50%. Further addition of CLOOH, and in particular 9-CLOH and 9/13-CLOH is effective in neutralising the effect of TRI 50c, such that the thrombin activity is significantly restored.

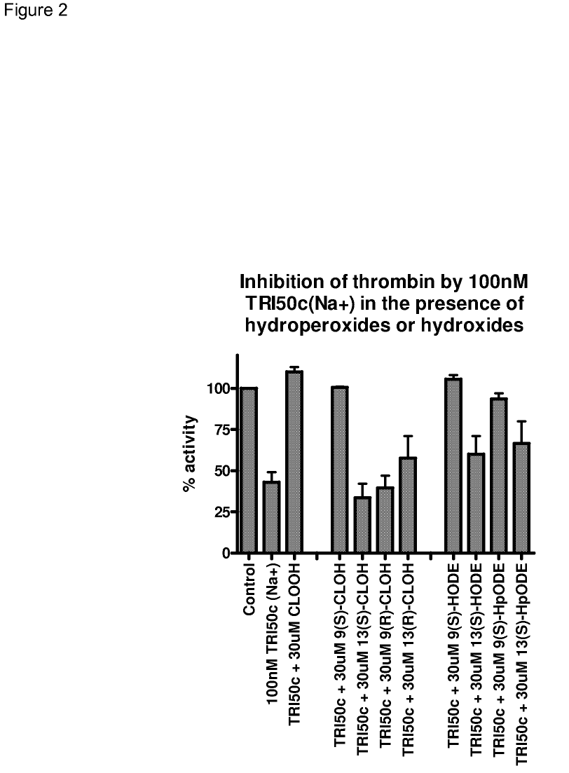

Referring to FIG. 2, the test procedures were applied also to various of the linoleic acids themselves. These further assays also present results for different stereoisomers of these materials: The nomenclature in FIG. 2 is:
9(S)-HODE —9-(S)— hydroxy-10E,12Z-octadecadienoic acid;
13(S)-HODE —13-(S)-hydroxyoctadecadienoic acid *;
9(S)-HpODE —9-(S)-hydroperoxy-10E,12Z-octadecadienoic acid;
13(S)-HpODE —13(S)-hydroperoxy-octadecadienoic acid.

Compound TRI 50b used in some of the examples is the pinacol ester of TRI 50c and is a prodrug for TRI 50c. TGN 255 is the monosodium salt of TRI 50c.

*It will be appreciated that materials such as 13(S)-HODE where hydroxy-substituted carbon is directly bonded to the C═C double bond may exist in the corresponding keto form and such forms are within the scope of the present disclosure.

From FIG. 2 it is apparent that particularly good TRI 50c neutralising activity is shown by CLOOH, 9(S)—CLOH, 9(S)-HODE and 13(S)—HpODE with beneficial effects being noted for other compounds also. In general, the (S) configuration is preferred in the compounds according to the disclosure, more especially so for HODE and HpODE, but the (R) configuration may also be useful.

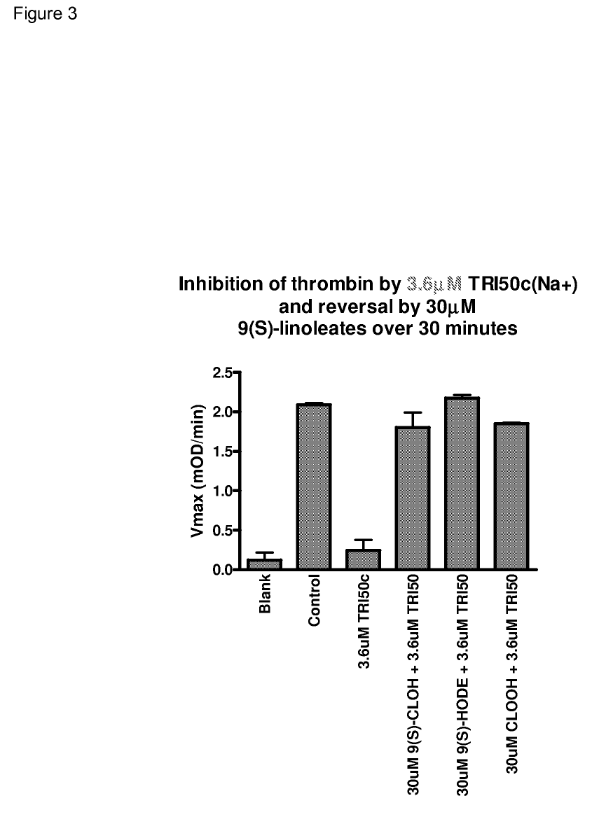

FIG. 3 summarises the neutralising ability of three particular 9(S)linoleates against TRI 50c over 30 minutes and shows that while the neutralising abilities of 9(S)—CLOH and 9(S)—CLOOH are very good, a particularly good effect is shown by 9(S)-HODE.

### Example 2

### IC₅₀ Values

50 μl thrombin (33.3 ng/ml in assay buffer) and 20 μl TRI 50c (300 nM final) were added to 10 μl assay buffer (100 mM Na orthophosphate (80% Na₂HPO₄ and 20% NaH₂PO₄), 200 mM NaCl, 0.5% PEG 6000, 0.02% Na azide, pH 7.5) and incubated for 5 minutes at 37° C. After the incubation period 100 μl of hydroxy fatty acid was added and further incubated for 30 minutes at 37° C. After the second incubation period, 20 μl of thrombin substrate (50 μM, S2238) was added and changes in Vmax was monitored on a plate reader for 10 minutes using a wavelength of 405 nm at 37° C. Results were expressed as percentage reversal of TRI 50c inhibition.

| | | IC₅₀ |
|---|---|---|
| Name | Structure | (μM) |
| 9(S)-HODE | 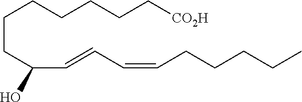 | 12 |
| 9(R)-HODE | 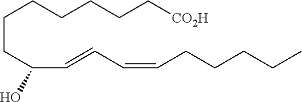 | IA |
| 9(S)-HODE cholesteryl ester | 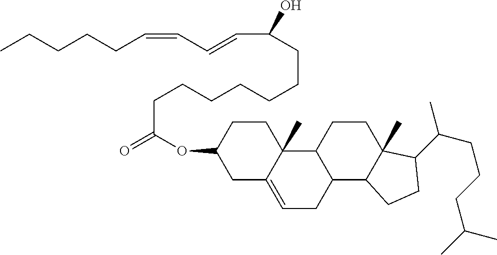 | 4.4 |
| 9(S)-HpODE | 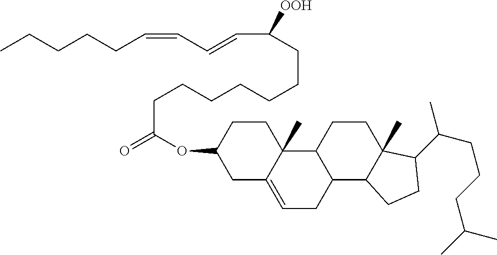 | >30 |
| 5(S)-HPETE | 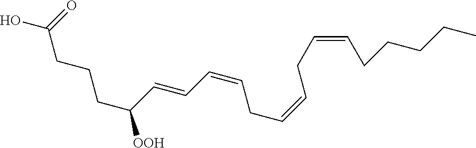 | 25 |
| (+/−)5-HETE | 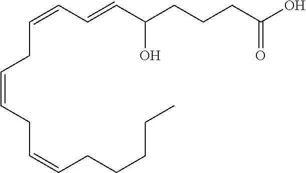 | >30 |
| 8(S)-HETRE | 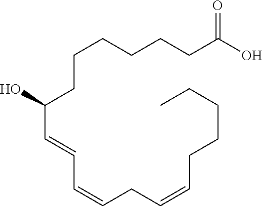 | 16 |
| 9(S)-HOTRE | 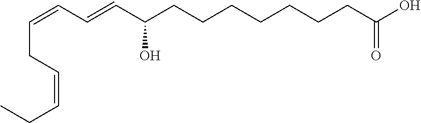 | >30 |
| (+/−)11-HEDE | 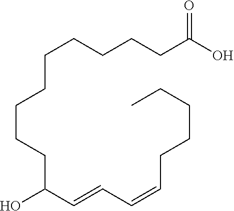 | >30 |
| (+/−)8-HETE | 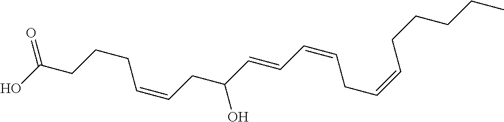 | >30 |
| 8(S)-HEPE | 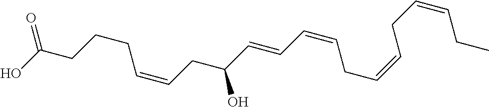 | 14 |
| (+/−)8,9-DIHETRE | 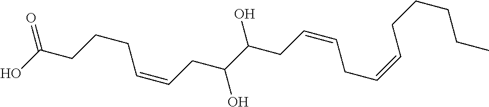 | >30 |
| 8(S), 15(S)-DIHETE | 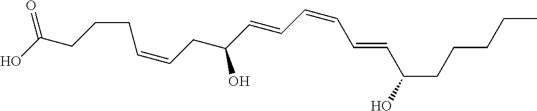 | >30 |
| 8(S), 15(S)-DIHETE* | 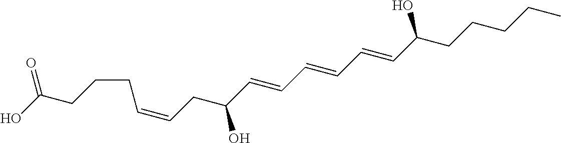 | >30 |
| Methyl 6- hydroxybicyclo[2.2.2]octane- 2-carboxylate | 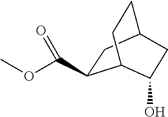 | >30 |
| (−)-Isoborneolacetic Acid | 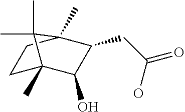 | >30 |
| (+)-Brefeldin A | 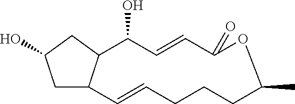 | >30 |

Key: IA = inactive under the specified conditions

### CONCLUSION

The data in this application indicate that the described compounds may be used therapeutically to neutralise the activity of biologically active organoboronate compounds. Particularly, the use of these products seems a viable therapeutic proposition to reverse therapy by TRI 50c or a TRI 50c salt or prodrug (e.g. TRI 50b) and it can also be predicted that other organoboronate compounds could be neutralised using this approach.

Thus, boronate inhibitors [Groziak M P. Boron therapeutics on the horizon. Am J Ther 2001; 8:321-8] have been described as proteasome inhibitors [Shah S A, Potter M W, McDade T P, et al. 26S Proteasome inhibition induces apoptosis and limits growth of human pancreatic cancer. J Cell Biochem 2001; 82:110-22; Hideshima T, Richardson P, Chauhan D, et al. The proteasome inhibitor PS-341 inhibits growth, induces apoptosis, and overcomes drug resistance in human multiple myeloma cells. Cancer Res 2001; 61:3071-6.] beta-lactamase inhibitors [Usher K C, Blaszczak L C, Weston G S, Shoichet B K, Remington S. Three-dimensional structure of AmpC beta-lactamase from Escherichia coli bound to a transition-state analogue: possible implications for the oxyanion hypothesis and for inhibitor design. Biochemistry 1998; 37:16082-92; Tondi D, Powers R A, Caselli E, et al. Structure-based design and in-parallel synthesis of inhibitors of AmpC beta-lactamase. Chem Biol 2001; 8:593-611.], dipeptidyl peptidase inhibitors[Tanaka S, Murakami T, Horikawa H, Sugiura M, Kawashima K, Sugita T. Suppression of arthritis by the inhibitors of dipeptidyl peptidase IV. Int J Immunopharmacol 1997; 19:15-24], inositol triphosphate receptor modulators, [Ma H T, Patterson R L, van Rossum D B, Birnbaumer L, Mikoshiba K, Gill D L. Requirement of the inositol trisphosphate receptor for activation of store-operated Ca2+ channels. Science 2000; 287:1647-51: Gregory R B, Rychkov G, Barritt G J. Evidence that 2-aminoethyl diphenylborate is a novel inhibitor of store-operated Ca²⁺ channels in liver cells, and acts through a mechanism which does not involve inositol trisphosphate receptors. Biochem J 2001; 354:285-90] antibacterials [Levy C W, Baldock C, Wallace A J, et al. A study of the structure-activity relationship for diazaborine inhibition of Escherichia coli enoyl-ACP reductase. J Mol Biol 2001; 309:171-80.] and antiestrogens[Endo Y, Yoshimi T, Yamakoshi Y. New estrogenic antagonists bearing dicarba-closo-dodecaborane as a hydrophobic pharmacophore. Chem Pharm Bull (Tokyo) 2000; 48:312-4.]. It may be predicted that these compounds will also be susceptible to control by this approach.

### Example 3

### Assay for Deboronation

This Example relates to a method for detecting the amount of deboronation of a product, TRI 50, in the presence of 9-HODE. A proposed mechanism for the degradation of TRI 50 is shown below.

### Mechanism:

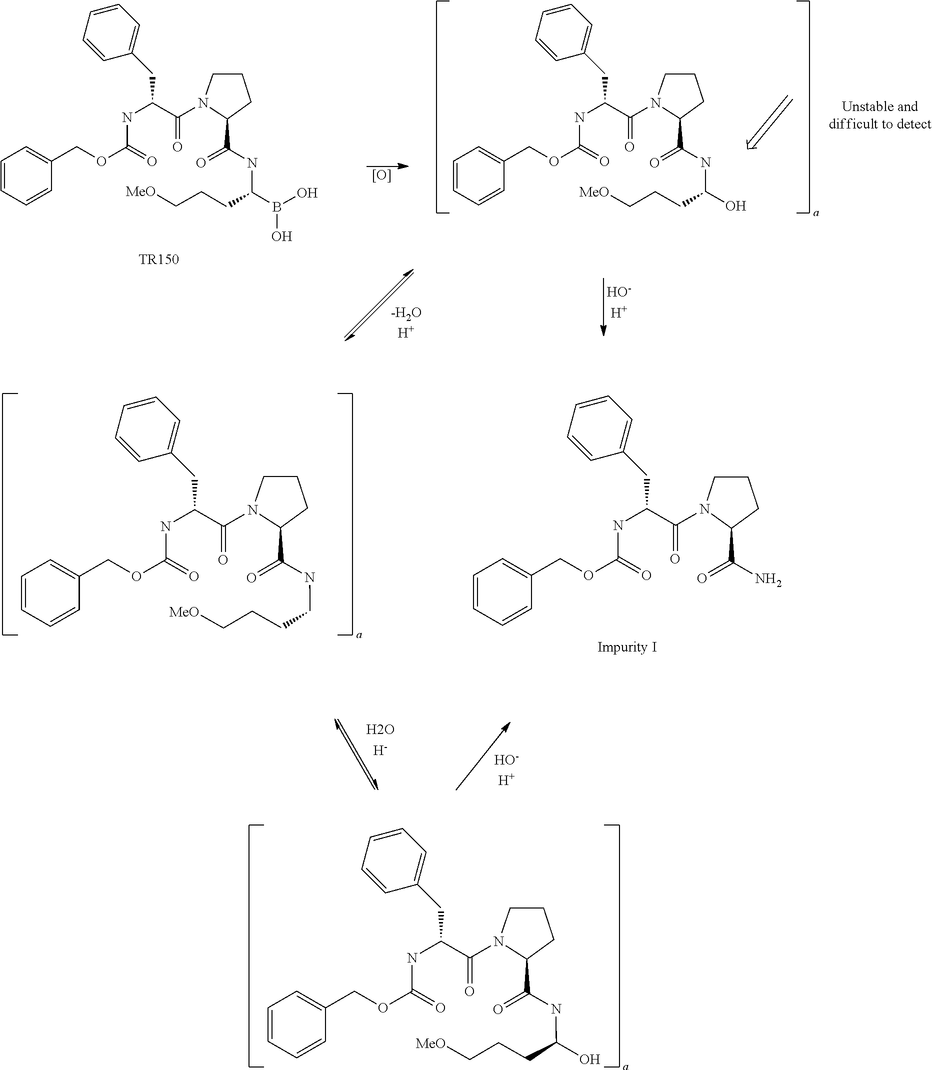

### Method

### Sample Preparation

### Preparation of Thrombin Buffer

The thrombin buffer was made up of 100 mM NaOphosphate [80% Na₂HPO₄ (11.36 g/l anhydrous)+20% NaH₂PO₄ (2.4 g/l anhydrous)], 200 mM NaCl (11.688 g/l), 0.5% PEG 6000 (5 g/l), 0.02% Na azide (0.2 g/l), pH 7.5.

### Preparation of TGN 255/9(S)-HODE Mixture

TGN 255 (20 mg) was dissolved in DMSO (1.9 ml). To this was added thrombin buffer (0.8 ml), followed by 9(S)-HODE (supplied by Cayman Chemical) in thrombin buffer (0.3 ml, 337 uM). The solution was incubated at 37° C. for 30 mins.

Total volume=3 ml

Concentration of TGN 255=6.7 mg/ml

Concentration of 9(S)-HODE=0.01 mg/ml

### LCMS Analysis

| | |
|---|---|
| Instrument: | Waters ZQ |
| Software: | MassLynx v.4.0 |
| Autosampler: | Waters 2767 |
| Detector: | Waters 2487 |
| Pump: | Waters 2525 |

### Gradient & Conditions

| | |
|---|---|
| Column: | Waters XTerra MS C-18, 5 u, 4.6 × 50 mm |
| Pump A: | Water + 0.1% Formic acid |
| Pump B: | Acetonitrile + 0.1% Formic acid |
| Injection volume: | 20 ul |
| Wavelength: | 254 nm |
| Ionisation mode: | Electrospray positive |

### Gradient:

| Time (min) | Flow (ml/min) | % A | % B |
|---|---|---|---|
| Initial | 1.50 | 95 | 5 |
| 20.00 | 1.50 | 5 | 95 |
| 25.00 | 1.50 | 5 | 95 |
| 25.50 | 1.50 | 95 | 5 |
| 30.00 | 1.50 | 95 | 5 |

### Preparation of Standards

Samples of TGN 255 (14 mg/ml in methanol) and Impurity I (5.6 mg/ml in methanol) were prepared as standards. The 9(S)-HODE assay sample was allowed to stand at room temperature overnight and analysed the following day.

### Results

The uv chromatogram for TGN 255/9(S)-HODE clearly shows the presence of Impurity I. The identity of the peak is confirmed by the corresponding mass. Due to the large excess of TGN 255 in the assay sample the Impurity I uv peak is significantly weaker in comparison. However, it is apparent that degradation has occurred, resulting in generation of Impurity I.

### Example 4

### Neutralisation by Cholesteryl Linoleate Hydroperoxide

Cholesteryl linoleate hydroperoxide (44 μM) was incubated with TRI-50b in the range 0 to 4.9 mM. Samples were taken at timed intervals and the residual TRI-50b concentration was determined by Method C (Materials and Methods). When pure cholesteryl linoleate hydroperoxide at a concentration of 44 μM was mixed with TRI-50b, a high rate of neutralisation was observed with an equivalence of approximately 1 mole/mole.

1. A method for therapeutically reducing or substantially destroying the activity of an organoboronate drug in a subject, comprising administering to the subject a therapeutically useful amount of a compound selected from organic acids which are other than boron acids and which comprise an aliphatic moiety substituted by a nucleophilic group, and salts and prodrugs of such acids.

2. The method of claim 1 wherein the acid contains from 12 to 30 carbon atoms.

3. The method of claim 1 wherein the nucleophilic group is a hydroxy, hydroperoxy or amino group.

4. The method of claim 1 wherein the aliphatic moiety contains a fragment —C*(H)A- where C* is a chiral centre of (S)-configuration and A is a said nucleophilic group.

5. The method of claim 4 wherein the acid contains a fragment —C(H)A- at the α-position to a carbon-carbon double bond.

6. The method of claim 5 wherein fragment —C(H)A- and the carbon-carbon double bond together form a fragment —C(H)A-CH═C(R^{a})(H) where R^{a} is an aliphatic moiety optionally substituted by one or more substituents selected from A groups, halogen, alkyl, alkoxy, haloalkyl and carboxy and wherein the double bond shown in the fragment is of trans (E) configuration and is optionally conjugated to a cis (Z) double bond within moiety R^{a}.

7. The method of claim 1 wherein the acid is of the formula (I): 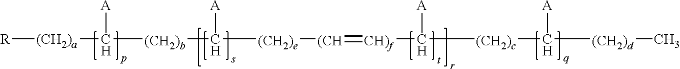
where:
R is —COOH, —P(O)(OH)₂, —HPOOH, or —SO₃H;
A is a nucleophilic group;
the sum of a and b is an integer from 4 to 9 inclusive and a or b may be 0;
the sum of c and d is an integer from 1 to 6 inclusive and c or d may be 0
e and f are independently 0 or 1;
r is an integer from 1 to 6; and
p, q, s and t are independently 0 or 1, the value of each instance of s and t being independent of the value of each other instance (if any) and there being at least one —C(H)A- moiety; and
at least one —CH₂— group within the compound may be replaced by an ether linkage —O— or an amine linkage —N—.

8. The method of claim 7 in which the acid comprises at least one —CH═CH— moiety directly bonded to a —C(H)A- moiety and wherein the —CH═CH— moiety is in trans (E) configuration.

9. The method of claim 7 in which the acid comprises a fragment —C(H)A-CH═CH— in which A is a hydroxy group

10. The method of claim 9 in which the hydroxy group is separated from the acid group (R in formula (I)) by 6 or 7 atoms.

11. The method of claim 7 in which a+p+b is at least 5 and the moiety R—(CH₂)a-[C(H)A]ₚ—(CH₂)_{b}— is directly bonded to a moiety C(H)A-CH═CH— in which a+p+b is from 5 to 10 and the double bond may be in trans (E) configuration, the double bond being conjugated to a double bond in cis (Z) configuration.

12. The method of claim 7 in which s and t are 0 in at least one fragment —[C(H)A]ₛ-(CH₂)ₑ—(CH═CH)_{f}-[C(H)A]ₜ— and p+q is 1.

13. The method of claim 7 in which f is 1 and e is either 0 or 1 in at least one fragment —[C(H)A]ₛ—(CH₂)ₑ—(CH═CH)_{f}-[C(H)A]ₜ— in which r is 2, 3 or 5.

14. The method of claim 7 in which the acid has from 14 to 20 carbon atoms and contains 1 or 2 nucleophilic groups A.

15. The method of claim 7 in which the acid has an R group which is —COOH or a salt or an ester thereof.

16. The method of claim 8 in which the or each A is —OH, —OOH or —NH₂ the identity of any one A group being independent from that of any other A groups.

17. The method of claim 7 in which the or each A is —OH.

18. The method of claim 1 in which the acid is of the Formula (II): 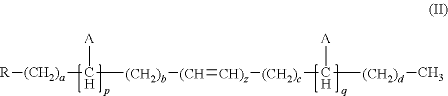
where:
R is —COOH, —P(O)(OH)₂, —HPOOH, or —SO₃H;
A is a nucleophilic group;
b is 0;
the sum of c and d is an integer from 1 to 6 inclusive and c or d may be 0
z is 1 or 2;
q is 0 or 1;
p is 1; and
in which the acid has a C═C moiety directly bonded to —[C(H)A]-.

19. The method of claim 1 in which the acid comprises a pharmacophore (or structural fragment) of Formula (III): 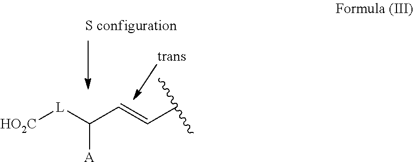
wherein:
A is a nucleophilic group; and
L is a linker containing from 5, 6, 7, 8, 9 or 10 in-chain atoms.

20. The method of claim 19 in which L contains 5, 6, 7 or 8 in-chain atoms.

21. The method of claim 19 in which A is hydroxy.

22. The method of claim 19 in which the in-chain atoms are C and in which the in-chain bonds are double or single bonds.

23. The method of claim 22 wherein the linker L is unsubstituted.

24. The method of claim 19 in which the linker is C₆ or C₇ alkylene or alkenylene.

25. The method of claim 22, wherein the linker is substituted and the substituent(s) are selected from A groups, halogen (e.g. F or Cl), 1C to 4C alkyl, 1C to 4C alkoxy, 1C to 4C haloalkyl and carboxy.

26. The method of claim 1 in which the acid comprises a pharmacophore of Formula (IV): 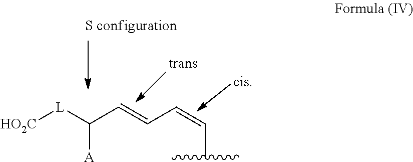
where
A is a nucleophilic group; and
L is a linker containing from 5, 6, 7, 8, 9, or 10 in-chain atoms.

27. The method of claim 1 in which the acid comprises a pharmacophore of formula (V) where the cis (Z) double bond is not conjugated to a second trans double bond: 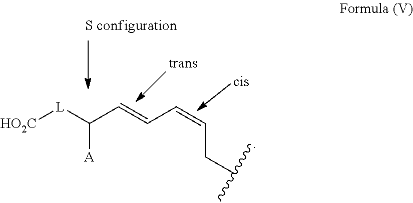
where
A is a nucleophilic group; and
L is a linker containing from 5, 6, 7, 8, 9, or 10 in-chain atoms.

28. The method of claim 1 wherein the acid is of the formula (XV): 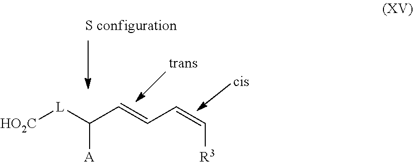
where R³ is a linear aliphatic moiety;
A is a nucleophilic group; and
L is a linker containing from 5, 6, 7, 8, 9 or 10 in-chain atoms.

29. The method of claim 1 in which the acid is of formula (VI): 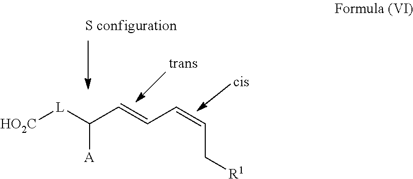
where
A is a nucleophilic group;
L is a linker containing from 5, 6, 7, 8, 9 or 10 in-chain atoms; and
R¹ is H or 1C-10C alkyl or 2C-10C alkenyl, optionally substituted by one or more substituents, e.g. 1, 2, 3 or 4 substituents selected from A groups, halogen (e.g. F or Cl), 1C to 4C alkyl, 1C to 4C alkoxy, 1C to 4C haloalkyl and carboxy.

30. The method of claim 29 in which R¹ is alkyl and has 3, 4, 5, 6, 7 or 8 carbon atoms.

31. The method of claim 29 in which R¹ is alkenyl and has one or more double bonds.

32. The method of claim 1 in which the acid is 9(S)-hydroxy-10E,12Z-octadecadienoic acid or a salt or prodrug thereof.

33. The method of claim 1 in which the acid is 8(S)-hydroxy-9E, 11Z, 14Z-eicosatrienoic acid or a salt or prodrug thereof.

34. The method of claim 1 in which the acid is 8(S)-hydroxy-5Z, 9E, 11Z, 14Z, 17Z-eicosapentaenoic acid or a salt or prodrug thereof.

35. The method of claim 28 where the acid is in the form of a salt or an ester.

36. The method of claim 1 wherein the boronate group (B(OH)₂) of the organoboronate drug is bonded to an sp³ carbon atom.

37. The method of claim 36 wherein the organoboronate drug is a peptide boronate whose C-terminal residue is of Boro-3-methoxypropylglycine.

38. The method of claim 37 wherein the drug is Cbz-(R)-Phe-(S)-Pro-(R)-Mpg-B(OH)₂, whether administered as the free acid, a salt or a prodrug, wherein Mpg-B(OH)₂ is a residue of an amino bornic acid of the formula H₂N—CH((CH₂)₃OMe-B(OH)₂.

39. An intravenous formulation comprising a compound as defined in claim 19.

40. The intravenous formulation of claim 39 wherein the compound is as defined in claim 28.

41. The intravenous formulation of claim 39 wherein the component is 9(S)-hydroxy-10E,12Z-octadecadienoic acid, or a prodrug or salt thereof.

42. The intravenous formulation of claim 39 wherein the acid is selected from 9(S)-hydroxy-10E,12Z-octadecadienoic acid, 8(S)-hydroxy-9E, 11Z, 14Z-eicosatrienoic acid or 8(S)-hydroxy-5Z, 9E, 11Z, 14Z, 17Z-eicosapentaenoic acid, substantially free of other isomers, or prodrugs or salts thereof.

43. A method for reversing the activity of a boropeptide drug in a subject, comprising administering to the subject a therapeutically useful amount of a hydroxy fatty acid or hydroperoxy fatty acid or salt or prodrug thereof.

44. A method of neutralising a boropeptidyl serine protease inhibitor comprising contacting said boropeptidyl serine protease inhibitor with a compound as defined in claim 1.
